(19) United States
(12) Patent Application Publication
(10) Pub. No.: US 2022/0120765 A1
(43) Pub. Date: Apr. 21, 2022

(54) MULTIPLEXED ASSAY AND METHODS OF USE THEREOF

(71) Applicant: Washington University, St. Louis, MO (US)

(72) Inventors: Melissa Budelier, St. Louis, MO (US); Randall Bateman, St. Louis, MO (US)

(21) Appl. No.: 17/422,051

(22) PCT Filed: Jan. 9, 2020
(86) PCT No.: PCT/US2020/012959

§ 371 Date: Jul. 9, 2021

Related U.S. Application Data
(60) Provisional application No. 62/790,290, filed on Jan. 9, 2019.

(51) Int. Cl. G01N 33/68; G16H 10/40; G16H 10/20

(52) U.S. Cl. CPC G01N 33/6896; G16H 10/40; G01N 2800/56; G01N 2333/4709; G01N 2800/2814; G16H 10/20

(57) ABSTRACT

The present disclosure provides methods for blood-based examination useful to identify subjects with Aβ amyloidosis and/or to identify subjects who should or should not undergo further testing or treatment for Aβ amyloidosis, as well as methods for treating subjects diagnosed with Aβ amyloidosis by the methods disclosed herein.

### FIELD OF THE TECHNOLOGY

The present disclosure relates to the use of a single assay for the detection of amyloidosis disorders using peripheral blood. In particular, the disclosure provides the use of a combination of markers of amyloid plaques (amyloid beta 42, amyloid beta 40), genetic risk (ApoE phenotype), and neurodegeneration (e.g. neurofilament light chain, tau and visinin-like protein one) into a single assay.

### BACKGROUND

The clinical diagnosis of an amyloidosis disorders typically relies on a history of slowly progressive cognitive impairment with early episodic memory deficits and ruling in or ruling out other potential causes of the disorder. In some clinical cases, amyloid PET and/or CSF biomarkers are used to evaluate for evidence of brain amyloidosis. In research settings, amyloid PET scans and/or CSF biomarkers are used to confirm brain amyloidosis in participants suspected to have Alzheimer disease dementia or to screen for individuals with preclinical Alzheimer disease (asymptomatic brain amyloidosis) for prevention trials. Unfortunately, both amyloid PET and CSF biomarkers have significant drawbacks including cost, availability and potential risks.

Thus, there is a need in the art for a single assay which increases efficiency while reducing analytical error, and financial burden, for the early detection of amyloidosis disorders, including the potential to predict a timeline to onset of Alzheimer disease.

### SUMMARY

In an embodiment, the present invention provides a method for identifying a subject as a candidate for further diagnostic testing and/or a therapeutic intervention, the method comprising: (a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and optionally determining the concentration of the marker of neurodegeneration; and (b) identifying the subject as a candidate further diagnostic testing and/or a therapeutic intervention when the Aβ42/Aβ40 value is less than 0.126, and the Aβ42/Aβ40 value is obtained by a system that provides a probability of detecting Aβ amyloidosis equal to or greater than about 90%.

In another embodiment, the present invention provides a method for screening subjects for a clinical trial for Aβ amyloidosis, the method comprising: (a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and determining the concentration of the marker of neurodegeneration; and (b) identifying the subject as a candidate for the clinical trial when the Aβ42/Aβ40 value is less than 0.126, and the Aβ42/Aβ40 value is obtained by a system that provides a probability of detecting Aβ amyloidosis equal to or greater than about 90%.

In another embodiment, the present invention provides a method to grade a subject for the stage or severity of disease, e.g. Aβ amyloidosis, the method comprising: (a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and optionally determining the concentration of the marker of neurodegeneration; and (b) identifying the subject as having or at risk of developing Aβ amyloidosis when the Aβ42/Aβ40 value is less than 0.126, and the Aβ42/Aβ40 value is obtained by a system that provides a probability of detecting Aβ amyloidosis equal to or greater than about 90%.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

FIG. 1 depicts the correspondence of baseline plasma and CSF Aβ42/Aβ40 with baseline amyloid PET. Baseline plasma (A) and CSF (B) Aβ42/Aβ40 were decreased in baseline amyloid PET-positive individuals. Receiver operating characteristic analyses demonstrate that baseline plasma (C) and CSF (D) Aβ42/Aβ40 were highly predictive of baseline amyloid PET status. The area under the curve (AUC) is noted with 95% confidence intervals. For the cut-offs listed, the positive percent agreement (PPA) and negative percent agreement (NPA) is provided with 95% confidence intervals. Baseline plasma (E) and CSF (F) Aβ42/Aβ40 were inversely correlated with baseline amyloid PET binding as measured on the centiloid scale. Baseline plasma and CSF Aβ42/Aβ40 were highly correlated (G). The Spearman rho (r) is noted with 95% confidence intervals for (E-G). Dashed red lines depict cut-offs for plasma or CSF Aβ42/Aβ40 based on the maximum Youden Index (A-G) or, for amyloid PET centiloid, the established cut-off for amyloid PET positivity (E-F).

FIG. 2 depicts the relationship of age, APOE ε4 status, and sex with baseline plasma and CSF Aβ42/Aβ40. Baseline plasma Aβ42/Aβ40 was lower with older age and was lower in APOE ε4 carriers and men (A). Baseline CSF Aβ42/Aβ40 was lower with older age and was lower in APOE ε4 carriers. Horizontal dashed red lines depict cut-offs for plasma or CSF Aβ42/Aβ40. Sloped lines represent the estimated Aβ42/Aβ40 as a function of age for the cross-sectional groups. Receiver operating characteristic analysis demonstrated a trend towards a higher area under the curve (AUC) for prediction of amyloid PET status when age and APOE ε4 status were included in the model (C). The AUC is noted with 95% confidence intervals. The combination of plasma Aβ42/Aβ40, age and APOE ε4 status were used to predict the likelihood of amyloid PET positivity (D).

FIG. 3 depicts the baseline plasma and CSF Aβ42/Aβ40 predict amyloid PET status conversion. Individuals who were amyloid PET-negative at baseline and converted to amyloid PET-positive over the follow-up period had lower baseline plasma Aβ42/Aβ40 than individuals who remained amyloid PET-negative (A). There was also a trend towards lower baseline CSF Aβ42/Aβ40 in amyloid PET converters versus non-converters (B). Dashed red lines depict cut-offs for plasma or CSF Aβ42/Aβ40. Individuals who were amyloid PET-negative at baseline with a positive plasma Aβ42/Aβ40 had a 12-fold greater risk of conversion to amyloid PET-positive over the follow-up period compared to individuals with a negative plasma Aβ42/Aβ40 (C). Individuals who were amyloid PET-negative at baseline with a positive CSF Aβ42/Aβ40 had a 5-fold greater risk of conversion to amyloid PET-positive over the follow-up period compared to individuals with a negative CSF Aβ42/Aβ40 (D).

FIG. 4 depicts the longitudinal change in plasma and CSF Aβ42/Aβ40. Both plasma (A) and CSF (B) Aβ42/Aβ40 declined within individuals over time. Thin lines connect values within an individual. The bolded rates are the average rates of change by linear regression for the entire longitudinal cohort and are represented by thick black lines. Dashed red lines depict cut-offs for plasma or CSF Aβ42/Aβ40 based on the analyses shown in FIG. 1. The rates of change for plasma and CSF Aβ42/Aβ40 for each individual were determined by linear regression and the slopes were plotted. The rate of change for plasma Aβ42/Aβ40 did not vary significantly by amyloid PET group (C). Amyloid PET converters had a faster decline in CSF Aβ42/Aβ40 compared to individuals who were amyloid PET-positive at both first and last time points (D). Dashed red lines depict a slope of zero (no change). Dotted lines are the average rate of change by linear regression for the entire longitudinal cohort.

FIG. 5 depicts the correlation of plasma and CSF Aβ42/Aβ40 with amyloid PET measures by PET tracer. Plasma (A-C) and CSF (D-F) Aβ42/Aβ40 were inversely correlated with measures of amyloid PET binding, regardless of the PET tracer used. Dashed red lines depict cut-offs depict established cut-offs for amyloid PET positivity. The Spearman rho (r) is noted.

FIG. 6 depicts extracted ion chromatograms from LC/MS of ApoE proteoforms (Baker-Nigh et. al. JBC, 2016) ApoE isoforms are each measured together on a single LC run across five minutes, capturing E2, 3, and 4 specific peptides, accurately identifying the ApoE geno/phenotype of an individual and quantifying the amount of ApoE. ApoE isoform type can be determined in as little as 10 mcl. Peptides indicated in the figure are SEQ ID Nos: 1-6.

FIG. 7 depicts ROC curve for A1342/40, APOE ε4 and age vs. Aβ42/40 in blood plasma. Comparison demonstrates the improved concordance of blood plasma with amyloid PET status by including age and ApoE. These measurements can be done on a single blood sample and a single mass spectrometry injection in 20 minutes and perform as well as or better than CSF (Gray et al, CSF Lumipulse tau/Aβ42 AUC=0.94, 2018).

FIG. 8 depicts evidence for plasma NfL as a useful marker for neurodegeneration.

FIG. 9 depicts VILIP-1 Plasma assay: Vilip-1 increases in stage from pathology normal, to amyloid plaque positive with very mild (CDR=0.5) or mild dementia (CDR=1).

FIG. 10 depicts a graph comparing 42/40 measured via the multiplex assay to 42/40 control. An analysis of the slope and R2 of a line forced through the origin resulted in y=1.0015x and R2=0.8807.

FIGS. 11A, B, and C depict graphs (FIGS. 11A and 11B) and a table (FIG. 11C) illustrating the correlation between the standard protocol and the multiplex protocol.

FIGS. 12A, 12B, 12C, and 12D depict graphs illustrating that ApoE genotype can be determined from 20% of an Abeta immunoprecipitation with 100% accuracy. FIG. 12A illustrates the detection of the E4 specific peptide (LGADMEDVR). Specific patient identifying numbers have been removed from the chart, but all samples were accurately determined with the assay. FIG. 12B illustrates the detection of the E2 and E3 specific peptide (LGADMEDVCGR). Specific patient identifying numbers have been removed from the chart, but all samples were accurately determined with the assay. FIG. 12C illustrates the detection of the E2 specific peptide (CLAVYQAGR; SEQ ID NO:7). Specific patient identifying numbers have been removed from the chart, but all samples were accurately determined with the assay. FIG. 12D illustrates the data normalized to total signal for the E2 specific peptide (CLAVYQAGR; SEQ ID NO:7). Specific patient identifying numbers have been removed from the chart, but all samples were accurately determined with the assay.

Various embodiments of the present invention will be described in detail with reference to the figures, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE INVENTION

The present invention relates to blood-based methods for detecting Aβ amyloidosis, and a system therefor. While Aβ42/Aβ40 ratios in the CSF are decreased by about 50% in the presence of Aβ amyloidosis, Aβ42/Aβ40 ratios in blood are decreased on average by 14% in amyloid positive subjects as compared to amyloid negative subjects. Importantly, the methods and systems described herein measure plasma concentrations of individual Aβ species with a high degree of precision. These precise measurements allow the small differences in plasma Aβ42 concentration between amyloid positive and amyloid negative subjects to be quantified accurately and therefore have clinical utility. Sensitivity and specificity of the amyloid-beta blood test substantially improve when ApoE status is determined. A single plasma based blood test analyzing Aβ42/40 along with ApoE status, increased the AUC from 88% to 95% relative to Aβ42/Aβ40 ratios alone. Moreover, the inclusion of one or more markers of neurodegeneration has the potential to more effectively aid in staging AD (e.g. asymptomatic years to symptom onset vs. mildly and moderately affected) and in monitoring response to therapeutics during clinical drug trials. Accordingly, the present invention also relates to methods to inform and direct clinical decisions including, but not limited to, conducting further diagnostic tests, enrolling a subject in a clinical trial, and initiating or continuing medical treatment. Other objects, advantages and features of the present invention will become apparent from the following description taken in conjunction with the accompanying figures.

The embodiments of this invention are not limited to particular method steps, which can vary and are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, wave length, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

The term “Aβ” refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically. For example, two such Aβ isoforms include Aβ40 and Aβ42; with the Aβ42 isoform being particularly fibrillogenic or insoluble and associated with disease states. The term “Aβ” typically refers to a plurality of Aβ species without discrimination among individual Aβ species. Specific Aβ species are identified by the size of the peptide, e.g., Aβ42, Aβ40, Aβ38 etc.

As used herein, the term “Aβ42/Aβ40 value” means the ratio of the concentration of Aβ42 in a blood sample obtained from a subject compared to the concentration of Aβ40 in the same blood sample.

As used herein, the term “Aβ42/Aβₓₓ value” means the ratio of the concentration of Aβ42 in a blood sample obtained from a subject compared to the concentration of another Aβ species in the same blood sample.

“Aβ amyloidosis” is clinically defined as evidence of Aβ deposition in the brain. A subject that is clinically determined to have Aβ amyloidosis is referred to herein as “amyloid positive,” while a subject that is clinically determined to not have A6 amyloidosis is referred to herein as “amyloid negative.” Aβ amyloidosis likely exists before it is detectable by current techniques. Nonetheless, there are accepted indicators of Aβ amyloidosis in the art. At the time of this disclosure, Aβ amyloidosis is typically identified by amyloid imaging (e.g., PiB PET, fluorbetapir, or other imaging methods known in the art) or by decreased cerebrospinal fluid (CSF) Aβ42 or a decreased CSF Aβ42/40 ratio. [¹¹C]PIB-PET imaging with mean cortical binding potential (MCBP) score >0.18 is an indicator of Aβ amyloidosis, as is cerebral spinal fluid (CSF) Aβ42 concentration of about 1 ng/ml by immunoprecipitation and mass spectrometry (IP/MS)). Values such as these, or others known in the art, may be used alone or in combination to clinically confirm Aβ amyloidosis. See, for example, Klunk W E et al. Ann Neurol 55(3) 2004, Fagan A M et al. Ann Neurol, 2006, 59(3), Patterson et. al, Annals of Neurology, 2015, 78(3): 439-453, or Johnson et al., J. Nuc. Med., 2013, 54(7): 1011-1013, each hereby incorporated by reference in its entirety. Subjects with A6 amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy, Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. patent Ser. Nos. 14/366,831, 14/523,148 and 14/747,453, each hereby incorporated by reference in its entirety. Additional methods are described in Albert et al. Alzheimer's & Dementia 2007 Vol. 7, pp. 170-179; McKhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as in individual for whom amyloid imaging may be clinically warranted. As a non-limiting example, a candidate for amyloid imaging may be a subject with one or more clinical signs of Aβ amyloidosis, one or more Aβ plaque associated symptoms, on one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer's disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “probability for detecting Aβ amyloidosis” refers to the extent to which detection is likely to occur, and is an indicator of the accuracy of a diagnostic test.

“ApoE” (NP_000032.1, UniProtKB Identifier P02649) is an apolipoprotein expressed from the APOE gene mapped to chromosome 19 (for example, the nucleotide sequence identified as GenBank Accession Number NM_000041, or NCBI Reference Sequence: NC_000019.10). ApoE has three major polymorphic forms: ApoE2 (Cys112, Cys158), ApoE3 (Cys112, Arg158), and ApoE4 (Arg112, Arg158). The ApoE2, ApoE3, and ApoE4 isoforms are encoded by the ε2, ε3 and ε4 alleles of the APOE gene. Unless expressly stated otherwise, “ApoE” refers to “human ApoE,” and includes functional fragments. “Recombinant ApoE” refers to ApoE encoded by a nucleic acid that has been introduced into a system (e.g. a prokaryotic cell, a eukaryotic cell, or a cell-free expression system) that supports expression of the nucleic acid and its translation into a protein. Methods for producing recombinant proteins are well-known in the art, and the production of recombinant ApoE disclosed herein is not limited to a particular system.

As used herein, the term “ApoE ε4 status” refers to the presence of the epsilon 4 allele on the apolipoprotein E gene. ApoE ε4 status may be determined at the nucleic acid level (e.g. sequencing the apolipoprotein E gene, etc.) or at the protein level (e.g. sequencing the ApoE protein, antibody-based methods, mass spectrometry based methods, etc.).

As used herein “markers of neurodegeneration” refers to biomarkers of neurodegenerative diseases or disorders such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease. Marks of neurodegeneration and methods of detecting the same are known in the art. Non-limiting examples of a marker of neurodegeneration include Tau, phosphorylated Tau, TDP-43, α-synuclein, SOD-1, FBP1, FUS, FKBP51, IRS-1, phosphorylated IRS-1, cathepsin D (CTSD), type 1 lysosome-associated membrane protein (LAMP1), ubiquitinylated proteins (UBP), heat-shock protein 70 (HSP70), neuron-specific enolase (NSE), neurofilament light chain (NFL), CD9, CD63, CD81, CD171, Visinin-like protein 1, BACE1, amyloid beta precursor protein, GHR, PD-1, APEX1, huntingtin, PRKN, and PSEN1.

“Neurofilament light chain” (NM_006158.4→NP_006149.2, UniProtKB Identifier P07196) comprises the axoskeleton and functions to maintain the neuronal caliber. Neurofilaments are type IV intermediate filament heteropolymers composed of light, medium, and heavy chains. They may also play a role in intracellular transport to axons and dendrites. Mutations in the neurofilament light chain (Nfl) gene cause Charcot-Marie-Tooth disease types 1F (CMT1F) and 2E (CMT2E), disorders of the peripheral nervous system that are characterized by distinct neuropathies. A pseudogene has been identified on chromosome Y. Nfl levels may be determined at the nucleic acid level (e.g. RT-PCR sequencing, etc.) or at the protein level (e.g., antibody-based methods, mass spectrometry based methods, etc.).

“Visinin-like protein 1” (NP_003376, UniProtKB Identifier P62760) is a protein that in humans is encoded by the VSNL1 gene. This gene is a member of the visinin/recoverin subfamily of neuronal calcium sensor proteins. The encoded protein is strongly expressed in granule cells of the cerebellum where it associates with membranes in a calcium-dependent manner and modulates intracellular signaling pathways of the central nervous system by directly or indirectly regulating the activity of adenylyl cyclase. Alternatively spliced transcript variants have been observed, but their full-length nature has not been determined.

As used herein, the term “ROC” means “receiver operating characteristic”. A ROC analysis may be used to evaluate the diagnostic performance, or predictive ability, of a test or a method of analysis. A ROC graph is a plot of sensitivity and specificity of a test at various thresholds or cut-off values. Each point on a ROC curve represents the sensitivity and its respective specificity. A threshold value can be selected based on an ROC curve to identify a point where sensitivity and specificity both have acceptable values, and this value can be used in applying the test for diagnostic purposes. If specificity only is optimized, then the test will be less likely to generate a false positive (diagnosis of the disease in more subjects who do not have the disease) at the cost of an increased likelihood that some cases of disease will not be identified (e.g. false negatives). If sensitivity is only optimized, the test will be more likely to identify most or all of the subjects with the disease, but will also diagnose the disease in more subjects who do not have the disease (e.g. false positives). A user is able to modify the parameters, and therefore select an ROC threshold value suitable for a given clinical situation, in ways that will be readily understood by those skilled in the art.

Another useful feature of the ROC curve is an area under the curve (AUC) value, which quantifies the overall ability of the test to discriminate between different sample properties, in this case to discriminate between those subjects with Aβ amyloidosis (i.e. amyloid positive) and those without Aβ amyloidosis (i.e. amyloid negative). A test that is no better at identifying true positives than random chance will generate a ROC curve with an AUC of 0.5. A test having perfect specificity and sensitivity (i.e., generating no false positives and no false negatives) will have an AUC of 1.00. In reality, most tests will have an AUC somewhere between these two values.

As used herein, the term “sensitivity” refers to the percentage of truly positive observations which is classified as such by a test, and indicates the proportion of subjects correctly identified as amyloid positive. In other words, sensitivity is equal to (true positive result)/[(true positive result)+(false negative result)].

As used herein, the term “specificity” refers to the percentage of truly negative observations which is classified as such by a test, and indicates the proportion of subjects correctly identified as amyloid negative. In other words, the percentage of healthy people who are correctly identified as not having a condition. Specificity is equal to (true negative result)/[(true negative result)+(false positive result).

In one embodiment, the range of the highest sensitivity is from 0.8 to 1. In another embodiment, the range of the highest specificity is from 0.8 to 1. In one embodiment, the range of the highest sensitivity is from 0.8 to 1 and the range of the highest specificity is from 0.8 to 1.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “healthy control group,” “normal group” or a sample from a “healthy” subject means a subject, or group subjects, who is/are diagnosed by a physician as not suffering from Aβ amyloidosis, or a clinical disease associated with Aβ amyloidosis (including but not limited to Alzheimer's disease) based on qualitative or quantitative test results. A “normal” subject is usually about the same age as the individual to be evaluated, including, but not limited, subjects of the same age and subjects within a range of 5 to 10 years.

As used herein, the term “blood sample” refers to a biological sample derived from blood, preferably peripheral (or circulating) blood. The blood sample can be whole blood, plasma or serum, although plasma is typically preferred.

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

### II. Method for Detection

One aspect of the present invention is a blood-based method for detecting Aβ amyloidosis, neurodegeneration, and/or for tau staging. Generally speaking, the method comprises detecting and quantifying the concentration of Aβ42, and optionally one other Aβ peptide, in a blood sample obtained from a subject, and comparing the Aβ42 concentration (or the Aβ42/Aβₓₓ value) to a predetermined threshold value. The method also generally comprises detecting ApoE and optionally a marker of neurodegeneration in the same blood sample and determining ApoE ε4 status and the concentration of the marker of neurodegeneration. Importantly, the methods described herein measure plasma concentrations of individual Aβ species and ApoE status with a high degree of precision. These precise measurements allow the small differences in plasma Aβ42 concentration between amyloid positive and amyloid negative subjects to be quantified accurately. Multiplexing Aβ42/Aβ40, ApoE phenotype and optionally a marker of neurodegeneration into a single assay provides a highly sensitive and specific concordance with detecting Aβ amyloidosis, while decreasing assay time. As a result, the method can be used to produce a system that has a probability for detecting Aβ amyloidosis equal to or greater than about 80%, equal to or greater than about 85%, equal to or greater than about 90%, preferably at least about 95%. Alternatively, or in addition, the system can be used to grade a subject for the stage of disease, e.g. Aβ amyloidosis, including identifying subjects most likely to develop Aβ amyloidosis. The inclusion of neurodegenerative markers into the assay has the potential to increase the specificity and sensitivity of the assay to aid in staging AD (e.g. asymptomatic years to symptom onset vs. mildly and moderately affected) and in monitoring response to therapeutics during clinical drug trials.

The method is not limited to a particular group of subjects. For example, the method may be incorporated into routine screening practices performed by general medical practitioners or specialists. In various other embodiments, a subject may be a participant in a clinical trial, a subject at risk of developing Aβ amyloidosis (e.g., due to known genetic, environmental, or lifestyle risks), a subject with at least one symptom of Aβ amyloidosis, a subject with a CAA associated symptom, or a subject initiating or continuing treatment for Aβ amyloidosis or a clinical disease associated with Aβ amyloidosis.

In embodiments that measure the concentration of Aβ42 and at least one other Aβ peptide (Aβₓₓ), the other Aβ peptide may be Aβ40, Aβ38, or any other Aβ peptide. In preferred embodiments, the other Aβ peptide is Aβ40 or Aβ38.

### (a) Blood Sample

A blood sample obtained from a subject is required. A blood sample may contain Aβ that is not modified to include a detectable label (“unlabeled Aβ”), or the sample may contain in vivo labeled Aβ. The term “in vivo labeled Aβ” refers to Aβ that was labeled in vivo following administration of label to a subject. Suitable labels are known in the art and include, but are not limited to, amino acids or amino acid precursors labeled with radioactive or non-radioactive isotopes. See, for example, US 20090142766 and US 20130115716, each hereby incorporated by reference in its entirety. Although in vivo labeling methods may increase the sensitivity of a detection method, an advantage of the present invention is that in vivo labeled Aβ is not required. In a preferred embodiment, the blood sample contains unlabeled Aβ. In another preferred embodiment, the blood sample does not contain in vivo labeled Aβ.

The blood sample should typically be large enough to allow the measurement of Aβ, ApoE and optionally a marker of neurodegeneration. A typical blood sample may be from about 0.5 ml to about 10 ml. More than one sample may be pooled for a particular time point. The blood sample may be collected directly as part of the method. Alternatively, a previously-obtained blood sample may be used. Methods of collecting a blood sample are well known in the art. For example, venipuncture, with or without a catheter, may be used to collect a blood sample. In another example, a finger stick, or the equivalent, may be used to collect a blood sample. Additives may or may not be added to the collected blood prior to plasma separation. Suitable additives include citrate, heparin, EDTA, Tween, and protease inhibitors.

### (b) Detecting and Quantifying Aβ, ApoE Peptides, and Markers of Neurodegeneration

The method of detecting and quantifying Aβ, ApoE, and optionally markers of neurodegeneration (e.g. Neurofilament light chain, Tau, and Visinin-like protein one) in a blood sample can and will vary but should be sensitive and precise enough to accurately quantify the concentration of Aβ, markers of neurodegeneration and APOE ε4 status in blood. A non-limiting measurement of assay precision is the coefficient of variation (CV). In some embodiments, the CV may be less than 5%. In some embodiments, the CV may be about 2-3%. Suitable methods are known in the art and include, but are not limited to, capture-specific assays, in particular antibody-based assays (e.g. ELISA, xMAP® technology, single molecule array (SIMOA™) technology, etc.), and high resolution mass spectrometry. Generally speaking, the method of detecting Aβ may also be used to quantify the concentration of Aβ and the method of detecting ApoE may be used to determine ApoE ε4 status. In some embodiments, quantification encompasses determining the Aβ42/Aβₓₓ value.

A blood sample, typically in the form of a plasma sample, may be used directly. Generally, however, additional processing of the sample occurs prior to analyzing the sample. In a preferred embodiment, one or more protease inhibitors are added to the sample. There are numerous commercial sources for protease inhibitors and protease inhibitor cocktails. In various embodiments, the blood sample may be aliquoted allowing the sample to be processed to detect Aβ, markers of neurodegeneration and ApoE in parallel. In such embodiments, the samples may be then pooled together prior to analysis.

In various other embodiments, additional techniques may be used to separate Aβ, markers of neurodegeneration and ApoE from other blood components (either partially or completely), or to concentrate the Aβ, markers of neurodegeneration and ApoE in a sample. As an example, immunoprecipitation may be used to partially or completely purify Aβ before it is analyzed. The immunoprecipitation antibody may be attached to a solid support, such as a bead or resin. Use of an antibody that binds to the mid-domain of Aβ can be used to immunoprecipitate multiple Aβ peptides, while selection of an antibody that binds to the N- or C-terminus of Aβ can be used to immunoprecipitate a subset of Aβ peptide(s). Protocols for immunoprecipitations are known in the art. Regarding the markers of neurodegeneration and ApoE, for example, immuno-enrichment or non-immuno-enrichment techniques may be used to separate and concentrate from other blood components. In one embodiment, an antibody-independent method of detecting and quantitating ApoE isoform-specific proteins is used. For example, PHM-Liposorb™ (Calbiochem, San Diego, Calif.), an absorbent typically used to remove lipids and lipoproteins from serum or plasma, may be used to capture ApoE from biological fluids.

Other methods of separating or concentrating Aβ, markers of neurodegeneration and ApoE may be used alone or in combination with. For example, chromatography techniques may be used to separate Aβ, markers of neurodegeneration or ApoE (or fragments thereof) by size, hydrophobicity or affinity. Aβ, markers of neurodegeneration and/or ApoE may also be cleaved into smaller peptides prior to detection. For instance, Aβ, markers of neurodegeneration and/or ApoE may be enzymatically cleaved with a protease to create several small peptides. Suitable proteases include, but are not limited to, trypsin, Lys-N, Lys-C, and Arg-N. In a preferred embodiment, Aβ may be enzymatically cleaved with Lys-N. In a preferred embodiment, ApoE may be enzymatically cleaved with trypsin.

In one embodiment, a capture-specific assay is used. Prior to analyzing the sample, one or more protease inhibitors are added to the sample. The sample, now containing one or more protease inhibitor(s), is then analyzed to determine the concentration of Aβ42. In certain embodiments, the concentration of at least one other Aβ peptide is also determined, for example Aβ40 and/or Aβ38. In a preferred embodiment, the capture-specific reagent of the assay is an antibody that is substantially Aβ-free.

In another embodiment, high-resolution tandem mass spectrometry is used. Prior to analyzing the sample, one or more protease inhibitors are added to the sample, the sample is aliquoted so Aβ, markers of neurodegeneration and ApoE can be detected in parallel. Then Aβ is immunoprecipitated using an anti-Aβ antibody, preferably an anti-Aβ antibody that specifically binds all targeted Aβ peptides. In parallel ApoE and optionally a marker of neurodegeneration is concentrated with or without immuno-enrichment. Following one or more wash steps, the concentrated peptides are proteolytically digested and the samples are pooled for analysis. Suitable proteases include, but are not limited to, trypsin, Lys-N, Lys-C, and Arg-N. Digestion may occur following elution or while the peptides are bound. Following one or more clean-up steps, digested peptides are analyzed by a liquid chromatography system interfaced with a high-resolution tandem MS unit (LC-MS/MS).

Additional processing of the sample may also occur prior to LC-MS/MS analysis. For example, the sample may be further processed following digestion by trichloroacetic acid (TCA) or trifluoroacetic acid (TFA) precipitation. Due to the high plasma protein to Aβ concentration ratio, and polymer and near-isobaric contamination (e.g., PEG or other buffer components), at the higher retention times that Aβ is detected by LC-MS/MS, accurate measurement of Aβ can be problematic when processing plasma. As a result, PEG and other contaminants cause ion suppression of Aβ peptides in the mass spectrometer. Beneficially, TCA or TFA precipitation can reduce such contamination. Alternatively, or in addition, the sample may be further processed following digestion (and optional TCA/TFA precipitation) with peracids, in non-limiting examples, performic acid (PFA), peracetic acid (PAA), pertrifluroacetic acid (PTFA) and such other peracids. This results in derivatization of Aβ to make it less hydrophobic and subsequently moving it away from the retention times of many hydrophobic contaminants.

In an exemplary embodiment, the mass spectrometry protocol outlined in the Examples is used.

### (c) Comparison to a Predetermined Threshold Value

Detection of Aβ amyloidosis occurs when the Aβ42 concentration (or Aβ42/Aβₓₓ value) in a blood sample obtained from a subject is lower than a predetermined threshold value that discriminates amyloid positive subjects from amyloid negative subjects, and when the predetermined threshold is obtained by a system that provides a probability of detecting Aβ amyloidosis equal to or greater than about 80%, equal to or greater than about 85%, equal to or greater than about 90%, preferably at least about 95%.

As used herein, the term “system” refers to the set of procedures used to determine a threshold value that discriminates amyloid positive subjects from amyloid negative subjects, including but not limited to the reagents, the assay used to detect and quantify Aβ, markers of neurodegeneration and ApoE, and the statistical methods used in the analysis. In addition, a system has either been validated to perform at a level that has a probability of detecting Aβ amyloidosis equal to or greater than 80%, or the system presently performs at said level even though validation has not been performed.

In one embodiment, the method for detecting and quantifying Aβ, ApoE and optionally a marker of neurodegeneration is selected from those disclosed in Section 11 (b), and the predetermined threshold value and probability of detecting Aβ amyloidosis is calculated by using a receiver operating characteristic (ROC) curve or other substantially similar method known in the art. For example, an ROC curve may be generated using the covariates Aβ42 concentration (or Aβ42/Aβₓₓ value), ApoE ε4 status, concentration of neurodegenerative marker and amyloid status (i.e. amyloid positive or amyloid negative) using blood samples obtained from amyloid positive or amyloid negative individuals of the same species as the subject. A plot is thus generated, which can be used to determine the sensitivity and specificity of various Aβ42 concentrations (or Aβ42/Aβₓₓ values), marker of neurodegeneration concentrations and ApoE status for predicting amyloid status. In certain embodiments, age may be used as another covariate. Area under the ROC curve may be used to evaluate the diagnostic accuracy. For example, an ROC AUC of 0.80 indicates there is an 80% probability that a randomly chosen individual with Aβ amyloidosis would have lower plasma Aβ42/Aβ40 value compared to a randomly chosen individual without Aβ amyloidosis. Various methods are known in the art for determining an optimal cut-off value that maximizes sensitivity and specificity to serve as a threshold for discriminating amyloid positive subjects. In one embodiment, the predetermined threshold is determined by a data point of the highest specificity at the highest sensitivity on the ROC curve. In another embodiment, the predetermined threshold is determined by a composit score via mathematical combination (for example logistical regression) of amyloid beta, ApoE isoform(s), and optionally one or more markers of neurodegeneration.

### III. Blood-Based Biomarker of Aβ Amyloidosis

Another aspect of the present invention is a blood-based biomarker of Aβ amyloidosis, wherein the blood-based biomarker is an Aβ42/Aβ40 value less than 0.130, determined by a system that provides a probability of detecting Aβ amyloidosis equal to or greater than about 80%, more preferably 85%. Stated another way, an Aβ42/Aβ40 value that can be used to identify an amyloid positive subject is an Aβ42/Aβ40 value less than 0.130.

In some embodiments, a blood-based biomarker of Aβ amyloidosis is an Aβ42/Aβ40 value less than about 0.128, preferably less than about 0.125. Alternatively, an Aβ42/Aβ40 value that can be used to identify an amyloid positive subject may be less than about 0.124, less than about 0.123, less than about 0.120, or less than about 0.117. In another example, an Aβ42/Aβ40 value that can be used to identify an amyloid positive subject may be less than about 0.115. In an exemplary embodiment, an Aβ42/Aβ40 value that indicates a subject is amyloid positive is an Aβ42/Aβ40 value of about 0.113 or less. In another exemplary embodiment, an Aβ42/Aβ40 value that indicates a subject is amyloid positive is an Aβ42/Aβ40 value of about 0.109 to about 0.113. In each of the above embodiments, the blood-based biomarker of Aβ amyloidosis described above can be optionally combined with an additional biomarker to further improve the diagnostic accuracy. When APOE ε4 status, and optionally age, are included with a plasma Aβ42/Aβ40 value in a system for detecting Aβ amyloidosis, the ROC AUC increased to 0.95 (0.91 to 0.98).

Another aspect of the present invention is a blood-based biomarker of Aβ amyloidosis, wherein the blood-based biomarker is an Aβ42/Aβₓₓ value, wherein Aβₓₓ is an Aβ peptide other than Aβ42. One of skill in the art will be able to determine values for other Aβ peptides based on the disclosures herein.

Methods for detecting and quantifying Aβ, ApoE and neurodegenerative marker peptides are known, and also described in Section II.

### IV. Methods for Identifying a Subject as a Candidate for Further Diagnostic Testing and/or Therapeutic Intervention

Another aspect of the present invention is a method for identifying or classifying a subject as a candidate for further diagnostic testing and/or for therapeutic intervention. The method comprises detecting and quantifying the concentration of Aβ42, one other Aβ peptide, ApoE and optionally a marker of neurodegeneration in a blood sample obtained from a subject, and identifying or classifying the subject as a candidate further diagnostic testing and/or therapeutic intervention when the subject tests positive for a blood-based biomarker of Section III or has a blood Aβ42 concentration (or a ratio of Aβ42 concentration to the concentration of another Aβ peptide) that is less than a predetermined threshold value, as described in Section II.

The method is not limited to a particular group of subjects. For example, the method may be incorporated into routine screening practices performed by general medical practitioners or specialists. In various other embodiments, a subject may be a participant or potential participant in a clinical trial, a subject at risk of developing Aβ amyloidosis (e.g., due to known genetic, environmental, or lifestyle risks), a subject with at least one symptom of Aβ amyloidosis, a subject with at least one CAA associated symptom. In some embodiments, the subject is a candidate for amyloid imaging.

It may be advantageous to use the methods disclosed herein to identify subjects in need of further diagnostic testing because the state-of-the-art test for Aβ amyloidosis, or diseases associated with Aβ amyloidosis, are limited by expense and availability, while the methods disclosed herein are minimally invasive and versatile. In some embodiments, a further diagnostic test is a cerebral spinal fluid (CSF) test to measure the concentration of one or more biomolecules found in the CSF. Non-limiting examples include one or more Aβ peptide, in particular Aβ42, tau, phospho-tau, neurofilament light chain, visinin-like protein one and ApoE. In other embodiments, a further diagnostic test is a neuroimaging test, such as a structural imaging test, a functional imaging test, or a molecular imaging test. Structural imaging tests are typically performed by magnetic resonance imaging (MRI) and/or computed tomography (CT) to provide information about the shape, position, or volume of brain tissue. Functional imaging tests are typically performed by positron emission testing (PET) and functional MRI (fMRI) to measure cellular activity in one or more regions of the brain. A non-limiting example of a functional imaging test is fluorodeoxyglucose (FDG)-PET. Molecular imaging tests use highly targeted radiotracers to detect cellular or chemical changes and are performed by technologies including PET, fMRI, and single photon emission computed tomography (SPECT). Non-limiting examples of a molecular imaging test include Pittsburgh compound B (PIB)-PET, florbetaben-PET, florbetapir-PET, and flutemetamol-PET.

The methods disclosed herein may also be used to identify subjects in need of therapeutic intervention. In some embodiments, therapeutic intervention may slow, inhibit or reverse amyloid deposition. Until such interventions advance from clinical trial stages, the methods disclosed herein may be used to identify subjects for enrollment in clinical trials and/or evaluate a subject's status during a clinical trial. In embodiments where a subject has one or more symptoms of Aβ amyloidosis, therapeutic intervention may slow or inhibit the worsening of the symptom and/or slow, inhibit, or prevent the onset of new symptoms.

### V. Methods for Treating a Subject with Aβ Amyloidosis

Another aspect of the invention is a method for treating a subject with a non-pharmacological treatment, a pharmacological treatment, or an imaging agent based on the subject's positive test result for a blood-based biomarker of Section III or the subject's blood Aβ42 concentration (or a ratio of Aβ42 concentration to the concentration of another Aβ peptide) and ApoE status as described in Section II.

In one embodiment, the method comprises measuring the Aβ42 concentration, ApoE status, and optionally a marker of neurodegeneration in a blood sample obtained from a subject, wherein the subject is diagnosed with Aβ amyloidosis when the Aβ42 concentration is less than a predetermined threshold value, wherein the Aβ42 concentration is compared to the subjects ApoE status and that discriminates amyloid positive subjects from amyloid negative subjects from subjects likely to develop Aβ amyloidosis. The predetermined threshold is obtained by a system that provides a probability of detecting Aβ amyloidosis equal to or greater than 80%, preferably at least about 85%; and administering a treatment to the diagnosed subject. The predetermined threshold value can be set as required by situational circumstances. For example, in certain clinical situations it may be desirable to minimize false-positive rates. These clinical situations may include, but are not limited to, the use of an experimental treatment (e.g., in a clinical trial) or the use of a treatment associated with serious adverse events and/or a higher than average number of side effects. Alternatively, it may be desirable to minimize false-negative rates in other clinical situations. Non-limiting examples may include treatment with a non-pharmacological intervention, the use of a treatment with a good risk-benefit profile, or treatment with a functional imaging agent, a molecular imaging agent (e.g., a radioimaging agent, etc.) followed by detection with PET, fMRI, SPECT, or the like. In certain embodiments, the method further comprises measuring the concentration of another Aβ variant (Aβxx) in the blood sample, wherein the subject is diagnosed with Aβ amyloidosis when the blood Aβ42/Aβxx value is less than a predetermined threshold value that discriminates amyloid positive subjects from amyloid negative subjects. In preferred embodiments, Aβxx is Aβ42, Aβ40, or Aβ38.

In another embodiment, the method comprises requesting a test that provides the results of an analysis determining whether the subject has an Aβ42 blood concentration less than a predetermined threshold value in view of the subjects ApoE status and optionally the concentration of a marker of neurodegeneration that discriminates amyloid positive subjects from amyloid negative subjects from subjects likely to develop Aβ amyloidosis, wherein the Aβ42 blood concentration was obtained by a system that provides a probability of detecting Aβ amyloidosis equal to or greater than 80%, preferably at least about 85%; diagnosing the subject with Aβ amyloidosis when the test results indicate the subject's Aβ42 blood concentration is less than a predetermined threshold value; and administering a treatment to the diagnosed subject. Requesting at test, as used herein, may refer to a physician requesting or ordering a test from a third party, from an in-house laboratory facility, or from a scientific lab capable of performing the test. The predetermined threshold value can be set as required by situational circumstances. For example, in certain clinical situations it may be desirable to minimize false-positive rates. These clinical situations may include, but are not limited to, the use of an experimental treatment (e.g., in a clinical trial) or the use of a treatment associated with serious adverse events and/or a higher than average number of side effects. Alternatively, it may be desirable to minimize false-negative rates in other clinical situations. Non-limiting examples may include treatment with a non-pharmacological intervention, the use of a treatment with a good risk-benefit profile, or treatment with a functional imaging agent, a molecular imaging agent (e.g., a radioimaging agent, etc.) followed by detection with PET, fMRI, SPECT, or the like. Alternatively, it may be desirable to maximize both sensitivity and specificity. In certain embodiments, the method further comprises requesting a test that provides the results of an analysis determining whether the patient has a blood Aβ42/Aβxx value less than a predetermined threshold value, in view of the subjects ApoE status, that discriminates amyloid positive subjects from amyloid negative subjects; and diagnosing the subject with Aβ amyloidosis when the test results indicated the subject's blood Aβ42/Aβxx value is less than a predetermined threshold value. In preferred embodiments, Aβxx is Aβ42, Aβ40, or Aβ38.

In another embodiment, the method comprises measuring the Aβ42 concentration and the Aβ40 concentration in a blood sample obtained from a subject, wherein the subject is diagnosed with Aβ amyloidosis when the calculated Aβ42/Aβ40 value is less than 0.126, as determined by a system that provides a probability of detecting Aβ amyloidosis equal to or greater than 80%, or optionally equal to or greater than about 85%; and administering a treatment to the diagnosed subject. In further embodiments, the Aβ42/Aβ40 value may be less than about 0.124, less than about 0.123, or less than about 0.120. In still further embodiments, the Aβ42/Aβ40 value may be less than about 0.117 or less than about 0.115. In still further embodiments, the Aβ42/Aβ40 value may be less about 0.113 or less. Alternatively, the Aβ42/Aβ40 value may be about 0.109 to about 0.113. The treatment may be a non-pharmacological treatment, a pharmacological treatment, or treatment with an imaging agent followed by detection of the imaging agent (e.g. with PET, fMRI, SPECT, or the like).

In another embodiment, the method comprises requesting a test that provides the results of an analysis determining whether the subject has an Aβ42/Aβ40 blood value less than 0.126, as determined by a system that provides a probability of detecting Aβ amyloidosis equal to or greater than 80%, or optionally equal to or greater than about 85%; diagnosing the subject with Aβ amyloidosis when the test results indicate the subject's blood Aβ42/Aβ40 value is less than 0.126, and administering a treatment to the diagnosed subject. In further embodiments, the Aβ42/Aβ40 value may be less than about 0.124, less than about 0.123, less than about 0.120, or less than about 0.117. In still further embodiments, the Aβ42/Aβ40 value may be less than about 0.115, or a value of about 0.113 or less. Alternatively, the Aβ42/Aβ40 value may be about 0.109 to about 0.113. The treatment may be a non-pharmacological treatment, a pharmacological treatment, or treatment with an imaging agent followed by detection with PET, fMRI, SPECT, or the like.

Non-limiting examples of non-pharmacological treatments include cognitive behavioral therapy, psychotherapy, behavioral management therapy, Montessori activities, memory training, massage, aromatherapy, music therapy, dance therapy, animal assisted therapy, and multi-sensory therapy. Non-limiting examples of pharmacological treatments include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.)

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A non-invasive, inexpensive screening test for Alzheimer disease (AD) is needed to advance clinical and prevention trials. An immunoprecipitation mass spectrometry (IPMS) blood test that is sensitive and specific for amyloid-β (Aβ) peptides Aβ42 and Aβ40 has been developed. In multiple cohorts, Aβ42/Aβ40 as measured by IPMS is significantly decreased in amyloid PET-positive individuals compared to amyloid PET-negative individuals. A logistic regression model for prediction of amyloid PET status by plasma Aβ42/Aβ40 had a ROC AUC of 0.88, which increased to 0.95 when APOE ε4 status and age were included in the model. A single assay for plasma Aβ42/Aβ40 and APOE ε4 status would reduce screening costs for enrolling participants in AD drug trials and has the potential to be used in clinical diagnosis.

An initial study (n=41 subjects, >500 samples) quantified plasma Aβ42 and Aβ40 in plasma and found that plasma Aβ42/Aβ40 was 14% lower in amyloid PET-positive individuals compared to amyloid PET-negative individuals. Receiver operating characteristic (ROC) analysis found that plasma Aβ42/Aβ40 distinguished amyloid PET status with an area under the curve (AUC) of 0.89 (Ovod, V. et al., Alzheimers Dement 13, 841-849 (2017)). Similar results were obtained in a different cohort with 158 participants (ROC AUC=0.88 [95% CI 0.82 to 0.93]). A subset of these participants (n=100) had a follow-up PET scan 2-9 years following the blood test. Individuals who were amyloid PET-negative at baseline with a low plasma Aβ42/Aβ40 (<0.1218) were 15-times more likely to become amyloid PET positive over the follow-up period, suggesting that plasma Aβ42/Aβ40 is sensitive to brain amyloidosis even below the threshold of amyloid-PET positivity. When APOE ε4 status and age were included with baseline plasma Aβ42/Aβ40 in a logistic regression model for baseline amyloid PET status, the ROC AUC increased to 0.95 (0.91 to 0.98). Given the improvement in prediction of brain amyloidosis when APOE ε4 status is considered, the possibility of performing both quantitation of plasma Aβ42 and Aβ40, as well as determination of APOE ε4 status via analysis of ApoE protein isoforms, with a single mass spectrometry assay was investigated.

100% concordance between ApoE genotype by sequencing and phenotype by LC-MS/MS was previous demonstrated (Baker-Nigh, A. et. al., J Biol Chem 291, 27204-27218 (2016)). After the addition of a set of 15N standards, Aβ was immunoprecipitated from plasma via a monoclonal anti-Aβ mid-domain antibody. ApoE analysis was performed in parallel without immuno-enrichment. The samples were endoproteolytically digested (Aβ with LysN and ApoE with trypsin), combined at the solid phase extraction step, and then co-analyzed by LC-MS/MS. Parallel (and/or Selected) reaction monitoring (PRM or SRM) of fragment ions enabled confident detection ApoE polymorphic peptides as well as the C-terminal Aβ42 and Aβ40 peptides. From these analyses, the Aβ42/Aβ40 ratio was calculated and ApoE proteotype assigned. Investigators were blinded during data acquisition and QC analysis. The data suggests that plasma Aβ42 and Aβ40 quantitation, and ApoE isoform analysis, can be determined by a single, sensitive, low cost mass spectrometry assay.

Thus, combining plasma Aβ42/Aβ40 value as measured by high precision IPMS with APOE ε4 status and age substantially improved sensitivity and specificity of the system to accurately detect brain-amyloidosis. We propose a single assay that allows both quantitation of plasma A642/A640 and determination of APOE ε4 status. This assay has the potential to improve early detection of AD and to substantially reduce the costs of recruiting a research cohort with brain amyloidosis for AD drug trials.

### Example 2

Participants enrolled in studies of memory and aging at Washington University were included in the study if they had undergone plasma collection within eighteen months of an amyloid PET scan. Because the assay used 1.6 ml of plasma, samples were selected for which the biorepository had sufficient plasma available. Participants of all ages and diagnoses were included, but the biorepository had greater availability of plasma from younger and cognitively normal participants. Therefore, this cohort represents a convenience sample. All participants underwent clinical assessments that included the Clinical Dementia Rating (CDR)¹⁴ and Mini-Mental State Examination (MMSE)¹⁵. APOE genotype was obtained from the Knight ADRC Genetics Core¹⁶. All procedures were approved by the Washington University Human Research Protection Office, and written informed consent was obtained from each participant.

CSF was collected as previously described¹⁷. Participants underwent LP at 8 am following overnight fasting. Twenty to thirty mis of CSF was collected in a 50 ml polypropylene tube via gravity drip using an atraumatic Sprotte 22-gauge spinal needle. The tube was gently inverted to disrupt potential gradient effects and centrifuged at low speed to pellet any cellular debris. CSF was aliquoted into polypropylene tubes and stored at −80° C. CSF Aβ42, tTau, and pTau were measured with the corresponding Elecsys immunoassays on the Roche cobas e601 analyzer¹⁸.

At the same session as CSF collection, blood was drawn into two 10 mL syringes pre-coated with 0.5 M EDTA, then transferred to two 15 mL polypropylene tubes containing 120 μl 0.5 M EDTA. The samples were kept on wet ice until centrifugation (<2 hours) to separate plasma from blood cells. The plasma was then transferred to a single 50 mL polypropylene tube, gently mixed, aliquoted into polypropylene tubes and stored at −80° C.

Targeted Aβ isoforms (Aβ38, Aβ40, and Aβ42) were simultaneously immunoprecipitated from 1.6 mL of plasma or 0.5 mL of CSF via a monoclonal anti-Aβmid-domain antibody (HJ5.1, anti-Aβ13-28) conjugated to M-270 Epoxy Dynabeads (Invitrogen, Carlsbad, Calif., USA). Prior to the plasma sample addition, assay tubes were pre-treated with 380 μL of a master mix containing 5.26× protease inhibitor cocktail (Roche, Basel, Switzerland), 0.263% (w/v) Tween-20, 2.63×PBS, and 2.63 M guanidine. After sample addition, plasma samples were spiked with 20 μL of a solution containing 3.75 pg/μL ¹²C¹⁵N-Aβ38, 25 pg/μL ¹²C¹⁵N-Aβ40, and 2.5 pg/μL ¹²C¹⁵N-Aβ42 (labeled peptides from RPeptide, Athens, Ga., USA) in 4:1 0.1% ammonium hydroxide:acetonitrile while CSF samples were spiked with 20 μL of a solution containing 75 pg/μL ¹²C¹⁵N-Aβ38, 500 pg/μL ¹²C¹⁵N-Aβ40, and 50 pg/μL ¹²C¹⁵N-Aβ42 in 4:1 0.1% ammonium hydroxide:acetonitrile. All subsequent immunoprecipitation steps were performed as previously described¹².

Human plasma analyses were performed as previously described¹². CSF analyses were performed on a Waters Xevo TQ-S triple quadrupole mass spectrometer interfaced with a Waters nanoAcquity chromatography system. For CSF analyses, extracted digests were reconstituted with 50 μl of 20 nM BSA Digest (Pierce, Appleton Wis., USA) in 10% formic acid/10% acetonitrile. A 4.5 μL aliquot of each reconstituted digest was loaded via direct injection onto a Waters 100×0.075 mm Acquity M-class HSS T3 column at 10% acetonitrile/2% dimethyl sulfoxide (DMSO)/0.1% formic acid with a flow rate of 600 nL/min for twelve minutes. After loading, peptides were resolved using an 8 minute linear gradient at 400 nL/min from 10% acetonitrile/2% DMSO/0.1% formic acid to 50% acetonitrile/2% DMSO/0.1% formic acid. The initial gradient was followed by a steeper linear gradient to 65% acetonitrile/2% DMSO/0.1% formic acid over 2 minutes at 400 nL/min. The column was then washed with 95% acetonitrile/2% DMSO/0.1% formic acid for 5 minutes 400 nL/min. Finally, the column was equilibrated back to initial solvent conditions for 5 minutes at 600 nL/min.

Peptides derived from human Aβ contained amino acids with the naturally-occurring ¹⁴Nitrogen (¹⁴N) isotope, while peptides derived from the exogenous Aβ spiked into samples as a standard contained amino acids that were uniformly labeled with ¹⁵Nitrogen (¹⁵N) isotope. The precursor/product ion pairs utilized for PRM (plasma) and SRM (CSF) analyses were chosen as previously described¹² and the derived integrated peak areas were analyzed using the Skyline software package¹⁹. For each Aβ isoform (Aβ40 or Aβ42) and its corresponding isotopomer (¹⁴N or ¹⁵N), integrated peak areas for selected product ions were summed. The Aβ42/Aβ40 ratio was calculated as follows: ((the sum of the integrated peak areas for ¹⁴N product ions for Aβ42/the sum of the integrated peak areas for ¹⁵N product ions for Aβ42) times the Aβ42 ¹⁵N calculated internal standard amount) divided by ((the sum of the integrated peak areas for ¹⁴N product ions for Aβ40/the sum of the integrated peak areas for ¹⁵N product ions for Aβ40) times the Aβ40 ¹⁵N calculated internal standard amount).

All mass spectrometry and quality control analyses were performed prior to sample unblinding. Values that failed quality control were not used if they did not meet threshold criteria for sample preparation (missing/mishandled samples), signal intensity, chromatographic properties (peak width/shape), coefficient of variation (technical replicates), and mass spectral noise. In total, 2.3% of (5 out of 216) plasma analyses and 3% (11 out of 361) of the CSF analyses failed the data QC process.

Plasma was collected from a cognitively normal young individual and an older individual known to have brain amyloidosis for use as high and low quality control (QC) calibrators, respectively. The high and low QC calibrators, along with intermediate mixes of the high and low QC calibrators, were run with every batch of plasma samples. Raw plasma Aβ42/Aβ40 values were normalized to the QC calibrators using linear regression to minimize batch-to-batch variability. This normalization was planned a priori because of observed batch effects in previous studies. Although high and low QC calibrators were also run with CSF samples, no significant batch-to-batch variability was noted and therefore no normalization was performed.

Amyloid PET was used as the reference standard for amyloidosis because it is a well-established biomarker and widely used in clinical trials for assessment of brain amyloid burden^{5,6,8}. Participants underwent a 60-minute dynamic scan with either ¹¹C Pittsburgh Compound B (PIB) or AV45. PET imaging was performed with a Siemens 962 HR+ ECAT PET or Biograph 40 scanner (Siemens/CTI, Knoxville Ky.). Structural magnetic resonance imaging (MRI) using MPRAGE T1-weighted images was also acquired and processed using FreeSurfer²⁰ (http://freesurfer.net/) to derive cortical and subcortical regions of interest²¹. Regional PIB or AV45 values were converted to standardized uptake value ratios (SUVRs) using cerebellar grey as a reference and partial volume corrected using a regional spread function approach²². Values from the left and right lateral orbitofrontal, medial orbitofrontal, precuneus, rostral middle frontal, superior frontal, superior temporal, and middle temporal cortices were averaged together to represent a mean cortical SUVR. Amyloid PET positivity was defined a priori with the established cut-offs of >1.42 for P1B²³ and >1.219 for AV45²⁴. Amyloid PET centiloid was used to combine PIB and AV45 data on a similar scale^{25,26}.

Characteristics of amyloid PET-positive and PET-negative groups were compared using T-tests for continuous variables and Chi-square tests for categorical variables. Receiver operating characteristic (ROC) analyses were performed to evaluate the correspondence between either plasma or CSF Aβ42/Aβ40 and amyloid PET status and were implemented with PROC LOGISTIC. Positive percent agreement (PPA) was defined as the percent of amyloid PET-positive individuals who were positive by a given plasma or CSF Aβ42/Aβ40 value. Negative percent agreement (NPA) was defined as the percent of amyloid PET-positive individuals who were negative by a given plasma or CSF Aβ42/Aβ40 value. The Youden index for each potential plasma or CSF Aβ42/Aβ40 value was calculated as the PPA plus the NPA minus one. The plasma or CSF Aβ42/Aβ40 value with the maximum Youden index was selected as the cut-off value and had the highest combined PPA and NPA, therefore best distinguishing between amyloid PET-positive and PET-negative individuals. Because amyloid PET centiloid values were not normally distributed, Spearman correlations were used to evaluate the relationship between amyloid PET centiloid and plasma or CSF Aβ42/Aβ40. Analysis of covariance with plasma or CSF Aβ42/Aβ40 as the outcome variable and centered age (age−the mean age for the cohort of 63.70 years), APOE ε4 status and sex as predictors were implemented with PROC GLM. Prediction of amyloid PET status of initially amyloid PET-negative individuals at their last PET scan based on baseline plasma or CSF Aβ42/Aβ40 status and follow-up time was implemented in PROC LOGISTIC. Survival analyses were implemented in PROC PHREG with the interval between the baseline plasma sample and the first positive amyloid PET scan used as the time to event. Linear regression was used to determine the rate of change; there was insufficient data to use linear mixed models.

For calculation of predicted savings in amyloid PET scans by screening with plasma Aβ42/Aβ40, the frequency of amyloid PET positivity as a function of age group and APOE ε4 status was estimated based on data from the A4 prevention study¹¹. The calculations assume that 35% of participants were APOE ε4 carriers, 76% were age 56-75 years old and 24% were 75-85 years old. The probability of a positive amyloid PET scan for individuals with a positive blood test was based on a logistic regression model generated with data from the present study with the blood test result (positive or negative), age (as a continuous variable) and APOE ε4 status as predictors.

Statistical analyses were conducted using SAS 9.4 (SAS Institute Inc., Cary, N.C.). Plots were created with GraphPad Prism version 6.07 (GraphPad Software, La Jolla, Calif.). Heat maps were generated with the R ggplot2 package. A p-value <0.05 was considered statistically significant. Data in the study will be deposited in the Washington University Knight Alzheimer Disease Research Center dataset and will be shared by request of any qualified investigator.

A total of 210 plasma samples from 158 individuals were analyzed (see Table 1 for participant characteristics) by immunoprecipitation-mass spectrometry (IPMS). 186 available CSF samples collected the same day as plasma from 145 individuals were assayed for Aβ42/Aβ40 by IPMS. Data on CSF Aβ42, tTau and pTau, as measured by Elecsys immunoassay, was available for 152 individuals.

TABLE 1
Baseline characteristics of all individuals with baseline plasma Aβ42/Aβ40
by amyloid PET status. Continuous measures are presented as the mean ± standard
deviation. The significance of differences between groups was determined by T-
tests for continuous variables and by Chi-Square tests for categorical variables.

| | Amyloid PET- | | Amyloid PET- | | |
|---|---|---|---|---|---|
| | negative | | positive | | |
| Characteristic | n= | | n= | | P= |
| Age at plasma collection | 115 | 60.8 ± 6.7 | 43 | 71.4 ± 6.8 | <0.0001 |
| (years) | | | | | |
| Sex (n, % Female) | 115 | 72, 63% | 43 | 30, 70% | N.S. |
| Years of education | 115 | 15.9 ± 2.2 | 43 | 15.2 ± 3.2 | N.S. |
| APOE ε4 status (n, % | 113 | 39, 35% | 43 | 27, 63% | 0.001 |
| carrier) | | | | | |
| CDR 0/0.5/1/2/3 (% >0) | 115 | 111/4/0/0/0 | 43 | 37/5/1/0/0 | 0.04 |
| | | (3%) | | (14%) | |
| MMSE (out of 30) | 115 | 29.4 ± 0.8 | 43 | 29.0 ± 1.6 | 0.02 |
| Plasma Aβ42/Aβ40 | 115 | 0.1276 ± 0.009 | 43 | 0.1152 ± 0.006 | <0.0001 |
| Amyloid PET centiloid | 115 | 1.0 ± 5.5 | 43 | 61.5 ± 32.6 | <0.0001 |
| AV45 SUVR | 27 | 0.91 ± 0.12 | 14 | 2.24 ± 0.64 | <0.0001 |
| PIB SUVR | 88 | 1.05 ± 0.10 | 29 | 2.26 ± 0.66 | <0.0001 |
| CSF Aβ42/Aβ40 | 105 | 0.1344 ± 0.0157 | 40 | 0.0768 ± 0.0164 | <0.0001 |
| Elecsys CSF Aβ42 (pg/ml) | 112 | 1272 ± 531 | 40 | 771 ± 297 | <0.0001 |
| Elecsys CSF tTau (pg/ml) | 112 | 177 ± 60 | 40 | 302 ± 111 | <0.0001 |
| Elecsys CSF pTau (pg/ml) | 112 | 15.7 ± 5.6 | 40 | 29.7 ± 13.1 | <0.0001 |

Abbreviations: Aβ40, amyloid-β 40; Aβ42, amyloid-β 42; CDR, Clinical Dementia Rating; CSF, cerebrospinal fluid; MMSE, Mini-Mental State Examination; N.S., not significant; PET, positron emission tomography; pTau, phosphorylated tau181; SUVR, standardized uptake value ratio; tTau, total tau.

An amyloid PET scan performed within eighteen months of the baseline plasma sample was negative for 115 individuals and positive for 43 individuals. The average interval between the plasma collection and the amyloid PET scan was 0.26±0.35 years (mean±standard deviation) with a range of 0 to 1.5 years. The age range extended from 46.1 to 86.9 years old. Compared to amyloid PET-negative individuals, individuals who were amyloid PET-positive were older (71.4±6.8 versus 60.8±6.7 years, p<0.0001), were more likely to carry an APOE ε4 allele (63% versus 35%, p=0.001), were more likely to have cognitive impairment as demonstrated by a clinical dementia rating greater than zero (14% versus 3%, p=0.04), and had lower CSF Aβ42 and higher CSF tTau and pTau (p<0.0001) by Elecsys immunoassays.

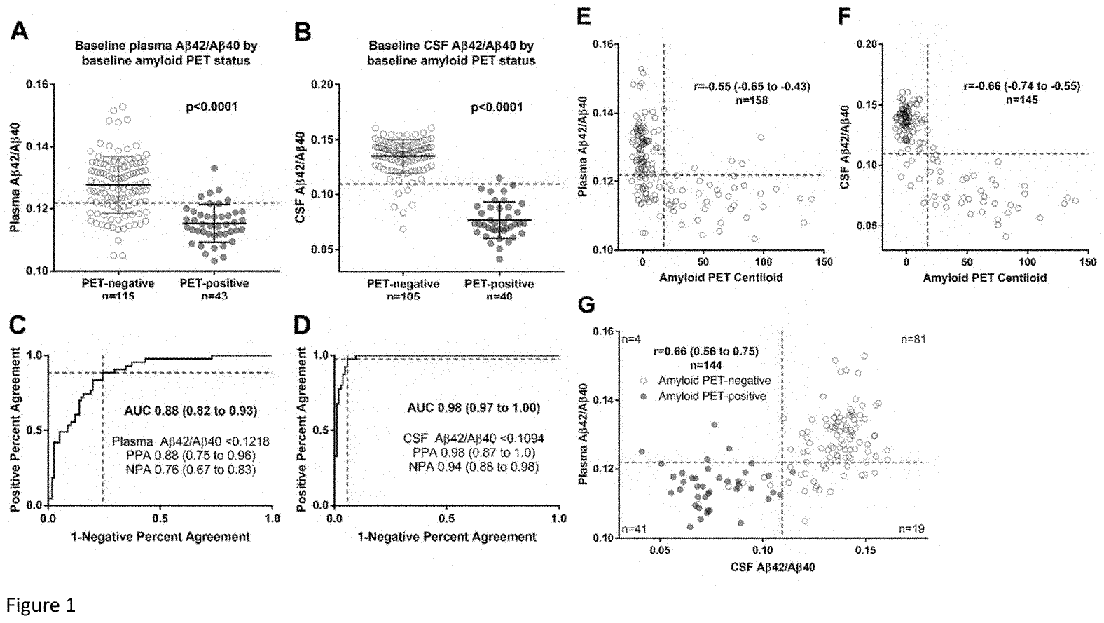

Individuals with a positive amyloid PET at baseline had a significantly lower baseline plasma Aβ42/Aβ40 compared to individuals with a negative amyloid PET at baseline (0.1152±0.006 versus 0.1276±0.0091, p<0.0001), (FIG. 1A). Receiver Operating Characteristic (ROC) analysis demonstrated that baseline plasma Aβ42/Aβ40 was a good predictor of baseline amyloid PET status, with an area under the curve (AUC) of 0.88 (95% confidence intervals [CI] of 0.82 to 0.93), (FIG. 1C). Alternative reference standards also showed high performance of the plasma Aβ42/Aβ40 assay: the ROC AUC was 0.85 (0.79 to 0.92) for a CSF Elecsys pTau/Aβ42 cut-off of 0.0198¹⁸ and 0.85 (0.78 to 0.92) for a CSF Elecsys pTau/Aβ42 cut-off of 0.0220²⁷. Our cohort represented a wide age range, but the performance of the assay was very similar in sub-cohort of individuals older than age 60 years (ROC AUC 0.87, 0.80 to 0.94). A plasma Aβ42/Aβ40 cut-off of <0.1218 was considered positive and had the maximum Youden Index with a positive percent agreement (PPA) of 0.88 (0.75 to 0.96) and a negative percent agreement (NPA) of 0.76 (0.67 to 0.83) with amyloid PET status (FIG. 1C). Baseline plasma Aβ42/Aβ40 was inversely correlated with amyloid PET on the continuous centiloid scale (FIG. 1E), with a Spearman rho of −0.55 (−0.65 to −0.43).

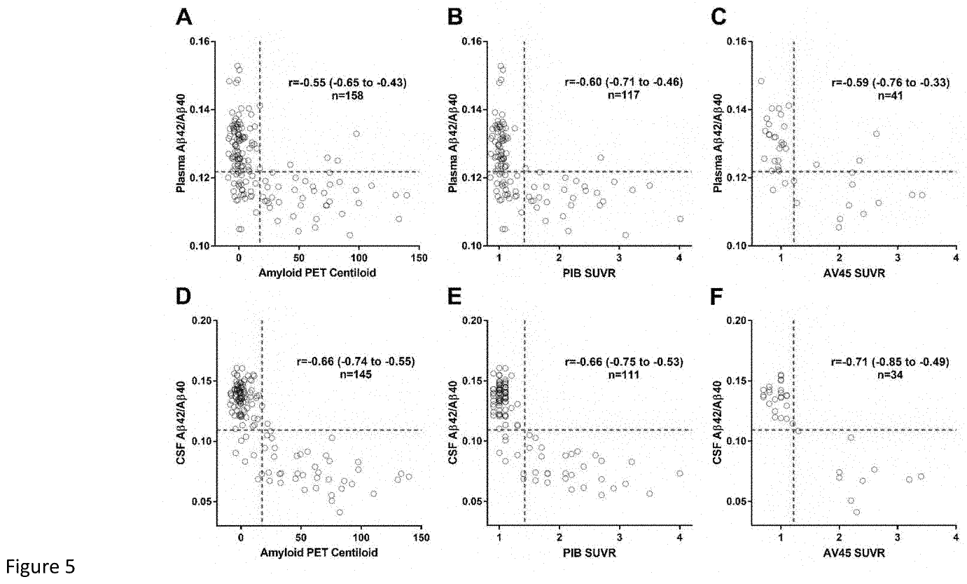

As expected, baseline IPMS CSF Aβ42/Aβ40 was also lower in individuals with a positive amyloid PET at baseline (FIG. 1B) and the concordance between CSF Aβ42/Aβ40 and amyloid PET was nearly perfect (FIG. 1D), with an AUC of 0.98 (0.97 to 1.0). A CSF Aβ42/Aβ40 cut-off of <0.1094 was considered positive and had the maximum Youden Index with a PPA of 0.98 (0.87 to 1.0) and an NPA of 0.94 (0.88 to 0.98). Baseline CSF Aβ42/Aβ40 was inversely correlated with amyloid PET centiloid (FIG. 1F), with a Spearman rho of −0.66 (−0.74 to −0.55). Similar inverse correlations between plasma and CSF Aβ42/Aβ40 and amyloid PET were obtained when the two tracers used, PIB and AV45, were evaluated separately (FIG. 5).

Baseline plasma and CSF Aβ42/Aβ40 were highly correlated (Spearman rho of 0.66, 0.56 to 0.75), (FIG. 1G). Using the cut-offs described herein, plasma and CSF Aβ42/Aβ40 had concordant predictions for amyloid status in 122 of 145 individuals (84%). All individuals with both a high CSF and plasma Aβ42/Aβ40 were amyloid PET-negative (n=81). 35 of 41 individuals with both a low plasma and CSF Aβ42/Aβ40 were amyloid PET-positive, but six were still PET-negative. Eighteen of nineteen individuals with a positive plasma Aβ42/Aβ40 but negative CSF Aβ42/Aβ40 were amyloid PET-negative. Four individuals with a negative plasma Aβ42/Aβ40 but positive CSF Aβ42/Aβ40 were amyloid PET-positive.

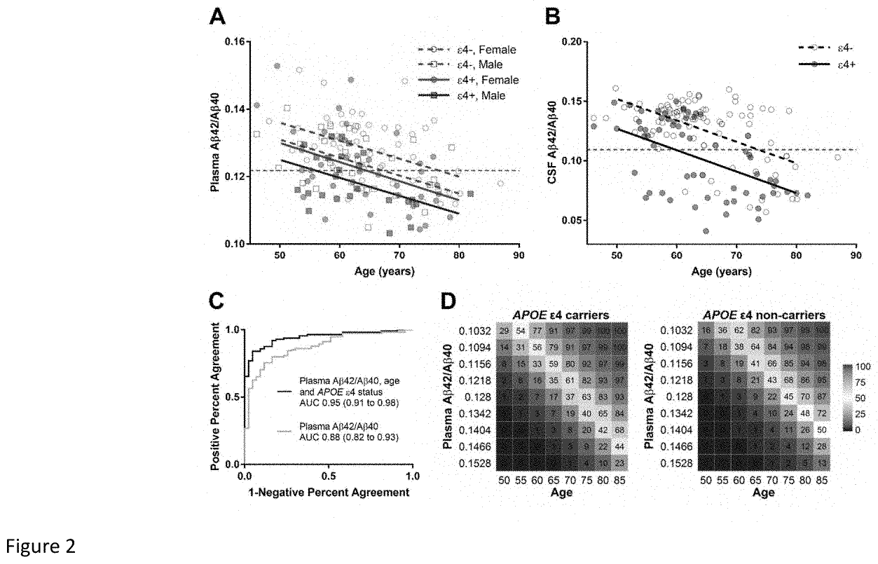

Baseline plasma Aβ42/Aβ40 was lower with older age (p<0.0001) and was lower in APOE ε4 carriers (p<0.0001) and men (p<0.002), (FIG. 2A and Table 2). There was no significant interaction between age and APOE ε4 status. Each decade of age, APOE ε4 carrier status and male sex was associated with lower plasma Aβ42/Aβ40 levels by ˜0.005 (for comparison, the difference between plasma Aβ42/Aβ40 in amyloid PET-positive and PET-negative individuals was ˜0.012). Similarly, baseline CSF Aβ42/Aβ40 was lower with older age and was lower in APOE carriers (both p<0.0001), (FIG. 2B and Table 2). In contrast to plasma Aβ42/Aβ40, CSF Aβ42/Aβ40 did not vary by sex.

TABLE 2
Relationship between plasma or CSF Aβ42/Aβ40 and age,
APOE ε4 status and sex. Centered age (age-63.70 years),
APOE ε4 status and sex were used as predictors of baseline
plasma and CSF Aβ42/Aβ40 values in analyses of covariance.
Baseline plasma Aβ42/Aβ40 was lower with older age,
in APOE ε4 carriers and men. Baseline CSF Aβ42/Aβ40
was lower with age and in APOE ε4 carriers but did not
vary by sex. The intercept is the estimated plasma or CSF
APOE ε4 at the mean age (63.70 years) for a female APOE
ε4 non-carrier. The estimates are the differences in the
plasma or CSF Aβ42/Aβ40 per year of age greater than
63.70 years, for APOE ε4 carriers and for men.

| | | | |
|---|---|---|---|
| Parameter | Estimate | S.E. | P= |

Age, APOE ε4 status and sex as predictors of plasma Aβ42/Aβ40

| | | | |
|---|---|---|---|
| Intercept | 0.1284 | 0.0010 | <0.0001 |
| Centered age (years) | −0.00055 | 0.000084 | <0.0001 |
| APOE ε4 carrier | −0.0061 | 0.0014 | <0.0001 |
| Male sex | −0.0046 | 0.0014 | 0.002 |

Age, APOE ε4 status and sex as predictors of CSF Aβ42/Aβ40

| | | | |
|---|---|---|---|
| Intercept | 0.1274 | 0.0030 | <0.0001 |
| Centered age (years) | −0.0018 | 0.00025 | <0.0001 |
| APOE ε4 carrier | −0.025 | 0.0041 | <0.0001 |
| Male sex | 0.0039 | 0.0042 | 0.36 |

Including age and APOE ε4 status with plasma Aβ42/Aβ40 in a model for prediction of amyloid PET status improved the ROC AUC from 0.88 (0.82 to 0.93) to 0.95 (0.91 to 0.98), although this difference did not reach significance (FIG. 2C). Sex was not a significant predictor in this model and did not improve the ROC AUC, likely because the model already correctly classified nearly all participants and sex did not improve classification of the remaining few discordant cases. The combination of plasma Aβ42/Aβ40, age, and APOE ε4 status were used to predict the likelihood of amyloid PET positivity (FIG. 2D).

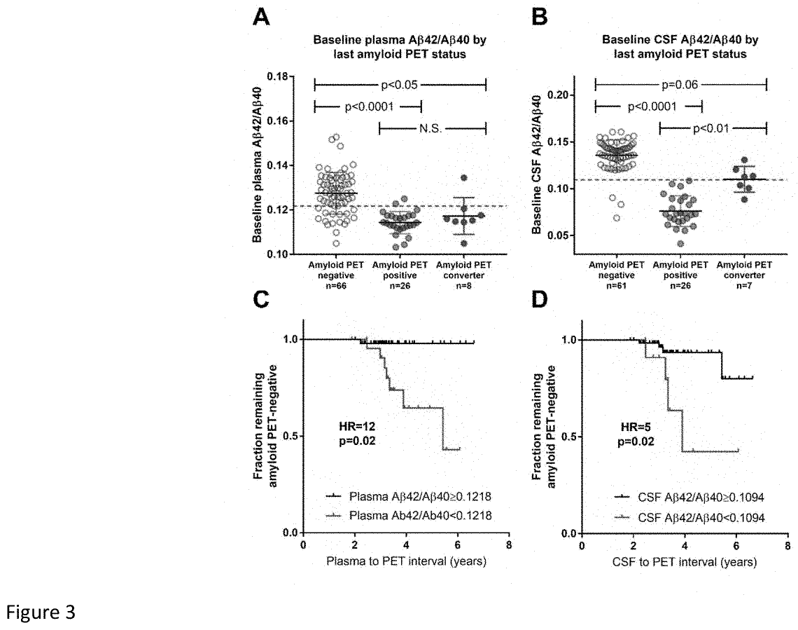

A sub-cohort of one-hundred individuals underwent at least one amyloid PET scan >1.5 years following their baseline plasma sample (for sub-cohort characteristics, see Table 4). For all subjects in this sub-cohort, the average interval between the baseline plasma collection and last amyloid PET scan was 3.9±1.4 years with a range of 1.9 to 9.0 years. 94 of these individuals also had matched CSF samples. Individuals who were amyloid PET-negative at baseline and converted to amyloid PET-positive over the follow-up period had lower baseline plasma Aβ42/Aβ40 than individuals who remained amyloid PET-negative (p<0.05, FIG. 3A). A logistic regression model that included follow-up time from plasma collection to the last PET scan found that plasma Aβ42/Aβ40 (continuous value) predicted conversion from amyloid PET-negative to amyloid PET-positive status (p=0.03) and predicted amyloid status at the last amyloid PET scan with a ROC AUC of 0.88, 0.81 to 0.95. A survival model for time to amyloid PET conversion demonstrated that individuals with a positive plasma Aβ42/Aβ40 (<0.1218) had a 12-fold higher risk of amyloid PET conversion than individuals with a negative plasma Aβ42/Aβ40 (p=0.02, FIG. 3C).

There was also a trend for amyloid PET converters to have a lower baseline CSF Aβ42/Aβ40 compared to individuals who remained amyloid PET-negative (p=0.06, FIG. 3B). A logistic regression model that included follow-up time from CSF collection to the last PET scan found that CSF Aβ42/Aβ40 (continuous value) predicted conversion from amyloid PET-negative to amyloid PET-positive status (p=0.007) and predicted amyloid status at the last amyloid PET scan with a ROC AUC 0.96, 0.92 to 1.00. Individuals who were amyloid PET-negative at baseline with a positive CSF Aβ42/Aβ40 (<0.1094) had a 5-fold greater risk of conversion to amyloid PET-positive over the follow-up period compared to individuals with a negative CSF Aβ42/Aβ40 (p=0.02, FIG. 3D).

Amyloid PET converters had a significantly higher baseline amyloid PET centiloid compared to individuals who remained amyloid PET-negative (6.9±4.7 versus −0.5±4.0, p<0.0001), suggesting amyloid PET converters had below-threshold brain amyloidosis. One individual classified as an amyloid PET converter with negative plasma and CSF Aβ42/Aβ40 at both the first and last time points had Elecsys CSF biomarkers that were inconsistent with brain amyloidosis (at the last time point CSF Aβ42 was 1434 pg/ml, tTau was 193 pg/ml and pTau was 17.5 pg/ml), suggesting that their last PET scan may be false positive.

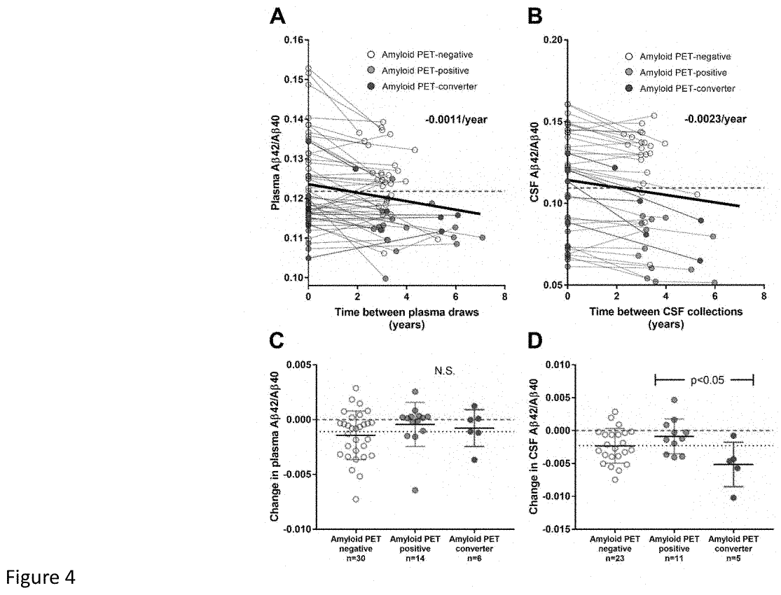

A sub-cohort of fifty individuals had longitudinal plasma Aβ42/Aβ40 collected within eighteen months of a longitudinal amyloid PET scan (FIG. 4, see Table 5 for participant characteristics), allowing examination of intra-individual rate of change. For all subjects in this sub-cohort, the average interval between the first and last plasma collections was 3.6±1.2 years with a range of 1.9 to 7.1 years. 39 of these individuals also had CSF samples that were analyzed for Aβ42/Aβ40. There was insufficient data to power linear mixed model analyses; instead, the intra-individual rate of change for each participant was estimated using linear regression. There was a significant decline in both plasma (−0.0011/year) and CSF Aβ42/Aβ40 (−0.0023/year) over time (p<0.001 and p<0.0001 by one-sample T-test, respectively). There was no difference in the rate of change of plasma Aβ42/Aβ40 by amyloid PET group (one-way ANOVA was not significant, FIG. 4C). However, amyloid PET converters had a faster decline in CSF Aβ42/Aβ40 compared to individuals who were amyloid PET-positive at baseline and the last time point (p<0.05 for one-way ANOVA, p<0.05 for Tukey's post-hoc test, FIG. 4D).

TABLE 3
Predicted savings in amyloid PET scans by using plasma Aβ42/Aβ40 as
a screen. The frequency of amyloid PET positivity as a function of age group
and APOE ε4 status was estimated based on data from the A4 prevention
study¹¹. The probability of a positive amyloid PET scan for individuals with
a positive blood test was based on a logistic regression model generated
with data from the present study with the blood test result (positive or
negative), age (as a continuous variable) and APOE ε4 status as predictors.

| | | | | | PET scans | |
|---|---|---|---|---|---|---|
| | | | Probability | PET scans | needed to | Percentage |
| | | | of amyloid | needed to | find 100 PET | of PET |
| | | | PET | find 100 PET | positive | scans |
| | | Amyloid | positive if | positive | participants | saved by |
| APOE | | PET | plasma | participants | if (plasma | using |
| ε4 | Age | positive | Aβ42/Aβ40 | (no blood | Aβ42/Aβ40 | blood test |
| status | (years) | rate | positive | test) | positive) | screening |
| ε4+ | 65-75 | 44% | 84% | 227 | 119 | 43% |
| | 75-85 | 68% | 98% | 147 | 102 | 31% |
| ε4− | 65-75 | 17% | 69% | 588 | 145 | 75% |
| | 75-85 | 27% | 95% | 370 | 105 | 71% |
| Overall | 65-85 | 30% | 80% | 333 | 125 | 62% |

TABLE 4
Baseline characteristics of individuals who contributed baseline plasma samples and longitudinal PET
data. Continuous measures are presented as the mean ± standard deviation. The significance of
differences between the amyloid PET-negative, stable group and the other two groups were determined
by T-tests for continuous variables and by Chi-Square or Fisher exact tests for categorical variables.

| | | | Converter from amyloid | | | | | |
|---|---|---|---|---|---|---|---|---|
| | Amyloid PET- | | PET-negative to PET- | | | | | |
| | negative, stable | | positive | | | Amyloid PET-positive, stable | | |
| Characteristic | n= | | n= | | p= | n= | | p= |
| Length of follow-up | 66 | 3.6 ± 1.1 | 8 | 5.2 ± 2.1 | <0.001 | 26 | 4.3 ± 1.7 | 0.01 |
| (years) | | | | | | | | |
| Number of PET scans, | 66 | 61/3/1/1 | 8 | 8/0/0/0 | N.S. | 26 | 25/1/0/0 | N.S. |
| 2/3/4/5 | | | | | | | | |
| Age at plasma | 66 | 59.5 ± 6.4 | 8 | 69.8 ± 5.1 | <0.0001 | 26 | 68.4 ± 6.4 | <0.0001 |
| collection (years) | | | | | | | | |
| Sex (n, % Female) | 66 | 42 (64%) | 8 | 5 (63%) | N.S. | 26 | 18 (69%) | N.S. |
| Years of education | 66 | 16.0 ± 2.2 | 8 | 16.4 ± 3.0 | N.S. | 26 | 15.6 ± 3.3 | N.S. |
| APOE ε4 status (n, % | 65 | 20 (31%) | 8 | 4 (50%) | N.S. | 26 | 18 (69%) | 0.001 |
| carrier) | | | | | | | | |
| CDR 0/0.5/1/2/3 (% >0) | 66 | 64/2/0/0/0 | 8 | 8/0/0/0/0 | N.S. | 26 | 25/1/0/0/0 | N.S. |
| | | (3%) | | | | | (4%) | |
| MMSE (out of 30) | 66 | 29.5 ± 0.8 | 8 | 29.4 ± 1.2 | N.S. | 26 | 29.2 ± 1.1 | N.S. |
| Plasma Aβ42/Aβ40 | 66 | 0.1275 ± 0.0094 | 8 | 0.1173 ± 0.0083 | <0.01 | 26 | 0.1143 ± 0.0050 | <0.0001 |
| Amyloid PET centiloid | 66 | −0.5 ± 4 | 8 | 6.9 ± 4.7 | <0.0001 | 26 | 49.4 ± 26.1 | <0.0001 |
| AV45 SUVR | 6 | 0.89 ± 0.10 | | | N.A. | 2 | 1.81 ± 0.76 | 0.01 |
| PIB SUVR | 60 | 1.03 ± 0.08 | 8 | 1.21 ± 0.10 | <0.0001 | 24 | 2.15 ± 0.57 | <0.0001 |
| CSF Aβ42/Aβ40 | 61 | 0.1359 ± 0.0164 | 7 | 0.1100 ± 0.0138 | <0.001 | 26 | 0.0760 ± 0.0163 | <0.0001 |
| Elecsys CSF Aβ42 | 66 | 1256 ± 561 | 8 | 1030 ± 437 | <0.0001 | 26 | 771 ± 233 | <0.0001 |
| (pg/ml) | | | | | | | | |
| Elecsys CSF tTau | 66 | 179 ± 61 | 8 | 219 ± 78 | N.S. | 26 | 290 ± 93 | <0.0001 |
| (pg/ml) | | | | | | | | |
| Elecsys CSF pTau | 66 | 15.9 ± 5.8 | 8 | 20.2 ± 7.1 | 0.05 | 26 | 28 ± 11.2 | <0.0001 |
| (pg/ml) | | | | | | | | |

Abbreviations: Aβ40, amyloid-β 40; Aβ42, amyloid-β 42; CDR, Clinical Dementia Rating; CSF, cerebrospinal fluid; MMSE, Mini-Mental State Examination; N.A., not applicable; N.S., not significant; PET, positron emission tomography; pTau, phosphorylated tau181; SUVR, standardized uptake value ratio; tTau, total tau.

TABLE 5
Baseline characteristics of individuals who contributed baseline and longitudinal plasma samples and amyloid
PET data. Continuous measures are presented as the mean ± standard deviation. The significance of
differences between the amyloid PET-negative, stable group and the other two groups were determined by
T-tests for continuous variables and by Chi-Square or Fisher exact tests for categorical variables.

| | | | Converter from amyloid | | | | | |
|---|---|---|---|---|---|---|---|---|
| | Amyloid PET- | | PET-negative to PET- | | | | | |
| | negative, stable | | positive | | | Amyloid PET-positive, stable | | |
| Characteristic | n= | | n= | | p= | n= | | p= |
| Length of follow-up (years) | 30 | 3.2 ± 0.6 | 6 | 4.2 ± 1.7 | 0.01 | 14 | 4.2 ± 1.5 | 0.002 |
| Number of samples, 2/3 | 30 | 30/0 | 6 | 6/0 | N.S. | 14 | 12/2 | N.A. |
| Age at plasma collection | 30 | 58.5 ± 6.5 | 6 | 68.9 ± 5.4 | <0.001 | 14 | 65.7 ± 6.9 | 0.002 |
| (years) | | | | | | | | |
| Sex (n, % Female) | 30 | 19, 63% | 6 | 4, 67% | N.S. | 14 | 10, 71% | N.S. |
| Years of education | 30 | 16.5 ± 1.8 | 6 | 16.2 ± 3.5 | N.S. | 14 | 16.6 ± 2.7 | N.S. |
| APOE ε4 status (n, % carrier) | 29 | 8, 28% | 6 | 3, 50% | N.S. | 14 | 10, 72% | 0.009 |
| CDR 0/0.5/1/2/3 (% >0) | 30 | 30/0/0/0/0 | 6 | 6/0/0/0/0 | N.S. | 14 | 14/0/0/0/0 | N.S. |
| | | (0%) | | (0%) | | | (0%) | |
| MMSE (out of 30) | 30 | 29.6 ± 0.6 | 6 | 29.2 ± 1.3 | N.S. | 14 | 29.1 ± 1.1 | N.S. |
| Plasma Aβ42/Aβ40 | 30 | 0.1288 ± 0.0109 | 6 | 0.1177 ± 0.0097 | 0.03 | 14 | 0.1149 ± 0.0037 | <0.0001 |
| Amyloid PET centiloid | 30 | −0.3 ± 4.4 | 6 | 7.5 ± 4.4 | 0.0003 | 14 | 44.9 ± 20.7 | <0.0001 |
| AV45 SUVR | 4 | 0.88 ± 0.09 | 0 | | N.A. | 0 | | N.A. |
| PIB SUVR | 26 | 1.03 ± 0.09 | 6 | 1.22 ± 0.10 | | 14 | 2.05 ± 0.46 | <0.0001 |
| CSF Aβ42/Aβ40 | 30 | 0.1338 ± 0.0201 | 6 | 0.1116 ± 0.0145 | 0.02 | 14 | 0.0778 ± 0.0119 | <0.0001 |
| Elecsys CSF Aβ42 (pg/ml) | 27 | 1138 ± 492 | 6 | 1082 ± 412 | N.S. | 14 | 803 ± 200 | 0.02 |
| Elecsys CSF tTau (pg/ml) | 27 | 163 ± 58 | 6 | 226 ± 81 | 0.03 | 14 | 259 ± 48 | <0.0001 |
| Elecsys CSF pTau (pg/ml) | 27 | 14.5 ± 5.4 | 6 | 20.5 ± 7.2 | 0.03 | 14 | 24.5 ± 4.8 | <0.0001 |

Abbreviations: Aβ40, amyloid-β 40; Aβ42, amyloid-β 42; CDR, Clinical Dementia Rating; CSF, cerebrospinal fluid; MMSE, Mini-Mental State Examination; N.A., not applicable; N.S., not significant; PET, positron emission tomography; pTau, phosphorylated tau181; SUVR, standardized uptake value ratio; tTau, total tau.

The value of using plasma Aβ42/Aβ40 to screen for individuals with a high risk of brain amyloidosis was evaluated (Table 3). The frequency of amyloid PET positivity as a function of age group and APOE ε4 status was based on data from the Anti-Amyloid Treatment in Alzheimer's (A4) prevention study, which included cognitively normal individuals aged 65-85 years¹¹. The probability of a positive amyloid PET scan for individuals with a positive blood test was based on a logistic regression model generated with data from the present study. By screening individuals with a positive plasma Aβ42/Aβ40, fewer confirmatory amyloid PET scans would be required to obtain a cohort of 100 individuals with a positive amyloid PET scan. The percentage of PET scans saved by first screening participant with plasma Aβ42/Aβ40 was highest in APOE ε4 non-carriers and younger individuals. For a cohort similar to A4, screening participants with plasma Aβ42/Aβ40 could reduce the number of amyloid PET scans required by approximately 62%.

This study provides Class I evidence that plasma Aβ42/Aβ40, as measured by a high precision immunoprecipitation and liquid chromatography-mass spectrometry assay, accurately diagnoses brain amyloidosis²⁸. It has previously been shown that individuals with brain amyloidosis have a decline in cognitive performance and a high rate of progression to AD dementia^{29,30}. In our study cohort comprised almost exclusively of cognitively normal individuals (94% with a CDR=0), we found high performance of plasma Aβ42/Aβ40 for detection of brain amyloidosis (ROC AUC 0.88), suggesting that plasma Aβ42/Aβ40 may be used as a screen for those at risk of AD dementia. Moreover, we found that individuals with a positive plasma Aβ42/Aβ40 but negative amyloid PET scan are at twelve-times the risk for converting to amyloid PET-positive (p=0.02). The sensitivity of the plasma Aβ42/Aβ40 assay to individuals who convert amyloid PET status suggests that plasma Aβ42/Aβ40 becomes positive earlier than the established amyloid PET threshold used for this study. Therefore, a positive plasma Aβ42/Aβ40 with a negative amyloid PET scan may represent early amyloidosis rather than a false positive result. Overall, our results demonstrate that plasma Aβ42/Aβ40, as measured by a high precision assay, could accurately detect brain amyloidosis in AD prevention drug trials that recruit cognitively normal research participants.

Many studies over the past two decades have evaluated plasma Aβ42 as a biomarker for Alzheimer disease, typically using immunoassays with relatively high variance and uncertain specificity, and overall found poor and inconsistent performance³¹. Recently, by using high precision assays, our group and others have found that plasma Aβ42/Aβ40 has a high correspondence to brain amyloidosis^{12,13}. In this study the difference between amyloid PET-positive and PET-negative individuals in this study was small, 0.1276±0.0091 versus 0.1152±0.006, but highly significant when measured with our high precision assay. The high accuracy of the assay is likely due to the high precision of mass spectrometry as an assay platform including the direct measurement of multiple specific Aβ species. Also, measuring both Aβ42 and Aβ40 in the same sample at the same time may reduce the variability introduced by measuring analytes with two separate assays.

We found that plasma Aβ42/Aβ40 levels were significantly associated with age, APOE ε4 status and sex. Recent studies using lower precision assays have found that plasma Aβ42/Aβ40 as measured by ELISA was associated with age and APOE ε4 status³² and that models including age and APOE ε4 status better predict amyloid status³³, but it is unclear whether these studies examined the relationship between sex and plasma Aβ42/Aβ40 levels. Interestingly, CSF Aβ42/Aβ40 levels were modulated by age and APOE ε4 status, but not sex. This dissociation suggests that plasma and CSF Aβ42/Aβ40 levels may be influenced by different factors. Other studies have explored factors that may modify plasma Aβ42/Aβ40^{32,34,35}, but further studies using high precision Aβ42/Aβ40 assays and larger cohorts are needed to clearly define these factors. Knowledge of factors that modify plasma Aβ42/Aβ40 can be used to improve models for prediction of brain amyloidosis. In our study using a high precision plasma Aβ42/Aβ40 assay, a model for prediction of amyloid PET status including plasma Aβ42/Aβ40, age and APOE ε4 status reached a ROC AUC of 0.95. Current CSF biomarker tests have approximately this level of correspondence with amyloid PET^{18,27}, suggesting that plasma Aβ42/Aβ40, especially when combined with other factors, may be accurate enough for clinical use at some point.

The most immediate use of the plasma Aβ42/Aβ40 assay is screening potential participants for Alzheimer's drug trials for brain amyloidosis. Age and APOE ε4 status could be used to improve the accuracy of the screen. If the plasma Aβ42/Aβ40 screen was positive, then a confirmatory test such as amyloid PET or CSF biomarkers may be performed, depending on the needs of the study. The plasma Aβ42/Aβ40 screen could significantly reduce or eliminate the number of confirmatory tests required to select a cohort of research participants with brain amyloidosis, especially in the case of prevention trials, which recruit cognitively normal individuals who have a relatively low rate of brain amyloidosis. We estimate that for a prevention trial similar to A4¹¹, pre-screening with plasma Aβ42/Aβ40 could reduce the number of amyloid PET scans required by 62%, which would result in substantially reduced time and costs for recruitment. If the plasma Aβ42/Aβ40 test combined with age and APOE ε4 status continues to demonstrate very high accuracy in diagnosis of brain amyloidosis (ROC AUC of ˜0.95), a single blood test including plasma Aβ42/Aβ40 and APOE genotype may be used for study inclusion without a need for confirmatory PET or CSF. The net effect would be acceleration of our progress towards an effective therapy for AD by decreasing time, cost and risk of drug trials, and one day enabling a blood test in the clinic to identify patients who could benefit from disease modifying treatment.

### REFERENCES

1. Hebert L E, Weuve J, Scherr P A, Evans D A. Alzheimer disease in the United States (2010-2050) estimated using the 2010 census. Neurology 2013; 80:1778-1783.
2. Blennow K, Hampel H, Weiner M, Zetterberg H. Cerebrospinal fluid and plasma biomarkers in Alzheimer disease. Nature reviews Neurology 2010; 6:131-144.
3. Witte M M, Foster N L, Fleisher A S, et al. Clinical use of amyloid-positron emission tomography neuroimaging: Practical and bioethical considerations. Alzheimer's & dementia 2015; 1:358-367.
4. O'Brien J T, Herholz K. Amyloid imaging for dementia in clinical practice. BMC medicine 2015; 13:163.
5. Klunk W E, Engler H, Nordberg A, et al. Imaging brain amyloid in Alzheimer's disease with Pittsburgh Compound-B. Annals of neurology 2004; 55:306-319.
6. Mattsson N, Carrillo M C, Dean R A, et al. Revolutionizing Alzheimer's disease and clinical trials through biomarkers. Alzheimer's & dementia 2015; 1:412-419.
7. Karran E, Hardy J. Antiamyloid therapy for Alzheimer's disease—are we on the right road? N Engl J Med 2014; 370:377-378.
8. Sperling R A, Rentz D M, Johnson K A, et al. The A4 study: stopping A D before symptoms begin? Science translational medicine 2014; 6:228fs213.
9. Carrillo M C, Brashear H R, Logovinsky V, et al. Can we prevent Alzheimer's disease? Secondary “prevention” trials in Alzheimer's disease. Alzheimer's & dementia: the journal of the Alzheimer's Association 2013; 9:123-131 e121.
10. Honig L S, Vellas B, Woodward M, et al. Trial of Solanezumab for Mild Dementia Due to Alzheimer's Disease. N Engl J Med 2018; 378:321-330.
11. Sperling R A, Donohue M, Raman R, et al. The Anti-Amyloid Treatment in Asymptomatic Alzheimer's Diseae (A4) Study: Report of Screening Data Results. Alzheimer's & dementia: the journal of the Alzheimer's Association 2018; 14:P215-P216.
12. Ovod V, Ramsey K N, Mawuenyega K G, et al. Amyloid beta concentrations and stable isotope labeling kinetics of human plasma specific to central nervous system amyloidosis. Alzheimer's & dementia: the journal of the Alzheimer's Association 2017; 13:841-849.
13. Nakamura A, Kaneko N, Villemagne V L, et al. High performance plasma amyloid-beta biomarkers for Alzheimer's disease. Nature 2018; 554:249-254.
14. Morris J C. The Clinical Dementia Rating (CDR): current version and scoring rules. Neurology 1993; 43:2412-2414.
15. Folstein M F, Folstein S E, McHugh P R. “Mini-mental state”. A practical method for grading the cognitive state of patients for the clinician. Journal of psychiatric research 1975; 12:189-198.
16. Pastor P, Roe C M, Villegas A, et al. Apolipoprotein Eepsilon4 modifies Alzheimer's disease onset in an E280A PS1 kindred. Annals of neurology 2003; 54:163-169.
17. Fagan A M, Mintun M A, Mach R H, et al. Inverse relation between in vivo amyloid imaging load and cerebrospinal fluid Abeta42 in humans. Annals of neurology 2006; 59:512-519.
18. Schindler S E, Gray J D, Gordon B A, et al. Cerebrospinal fluid biomarkers measured by Elecsys assays compared to amyloid imaging. Alzheimer's & dementia: the journal of the Alzheimer's Association 2018.
19. Pino L K, Searle B C, Bollinger J G, Nunn B, MacLean B, MacCoss M J. The Skyline ecosystem: Informatics for quantitative mass spectrometry proteomics. Mass Spectrom Rev 2017.
20. Fischl B, van der Kouwe A, Destrieux C, et al. Automatically parcellating the human cerebral cortex. Cerebral cortex 2004; 14:11-22.
21. Su Y, D'Angelo G M, Vlassenko A G, et al. Quantitative analysis of PiB-PET with FreeSurfer ROls. PloS one 2013; 8:e73377.
22. Su Y, Blazey T M, Snyder A Z, et al. Partial volume correction in quantitative amyloid imaging. NeuroImage 2015; 107:55-64.
23. Vlassenko A G, McCue L, Jasielec M S, et al. Imaging and cerebrospinal fluid biomarkers in early preclinical alzheimer disease. Annals of neurology 2016; 80:379-387.
24. Mishra S, Gordon B A, Su Y, et al. AV-1451 PET imaging of tau pathology in preclinical Alzheimer disease: Defining a summary measure. NeuroImage 2017; 161:171-178.
25. Klunk W E, Koeppe R A, Price J C, et al. The Centiloid Project: standardizing quantitative amyloid plaque estimation by PET. Alzheimer's & dementia: the journal of the Alzheimer's Association 2015; 11:1-15 e11-14.
26. Su Y, Flores S, Hornbeck R C, et al. Utilizing the Centiloid scale in cross-sectional and longitudinal PiB PET studies. Neuroimage Clin 2018; 19:406-416.
27. Hansson O, Seibyl J, Stomrud E, et al. CSF biomarkers of Alzheimer's disease concord with amyloid-beta PET and predict clinical progression: A study of fully automated immunoassays in BioFINDER and ADNI cohorts. Alzheimer's & dementia: the journal of the Alzheimer's Association 2018; 14:1470-1481.
28. Gross R A, Johnston K C. Levels of evidence: Taking Neurology to the next level. Neurology 2009; 72:8-10.
29. Vos S J, Xiong C, Visser P J, et al. Preclinical Alzheimer's disease and its outcome: a longitudinal cohort study. The Lancet Neurology 2013; 12:957-965.
30. Donohue M C, Sperling R A, Petersen R, et al. Association Between Elevated Brain Amyloid and Subsequent Cognitive Decline Among Cognitively Normal Persons. JAMA 2017; 317:2305-2316.
31. Olsson B, Lautner R, Andreasson U, et al. CSF and blood biomarkers for the diagnosis of Alzheimer's disease: a systematic review and meta-analysis. The Lancet Neurology 2016; 15:673-684.
32. Nakamura T, Kawarabayashi T, Seino Y, et al. Aging and APOE-epsilon4 are determinative factors of plasma Abeta42 levels. Ann Clin Transl Neurol 2018; 5:1184-1191.
33. Verberk I M W, Slot R E, Verfaillie S C J, et al. Plasma Amyloid as Prescreener for the Earliest Alzheimer Pathological Changes. Annals of neurology 2018.
34. Toledo J B, Vanderstichele H, Figurski M, et al. Factors affecting Abeta plasma levels and their utility as biomarkers in ADNI. Acta Neuropathol 2011; 122:401-413.
35. Toledo J B, Shaw L M, Trojanowski J Q. Plasma amyloid beta measurements—a desired but elusive Alzheimer's disease biomarker. Alzheimers Res Ther 2013; 5:8.

### Example 3

Due to the lack of a specific, simple and inexpensive test, current clinical diagnosis of Alzheimer's disease (AD) relies on progressive memory decline and cognitive impairment. However, clinical diagnosis has both poor sensitivity and specificity for AD and other neurodegenerative dementias. Current diagnostic tests in development include CSF and PET scans for tau tangle and amyloid-plaque pathologies. However, these approaches are invasive, require significant training, and are expensive. Further, early asymptomatic detection of AD pathology is necessary for enrollment of participants in research studies, clinical trials and prevention trials.

A blood test to improve clinical diagnosis and accelerate therapeutic development needs to have improved specificity and sensitivity in participants across a range from normal to cognitively impaired, be minimally invasive, and inexpensive. Further, the test should include measures of the key domains of AD: genetic risk (ApoE), pathology of amyloid plaques and tau tangles, and neurodegeneration to help determine the AD stage of individuals. A single test reporting these domains would capture the major aspects of AD and be useful in a dementia clinic. Towards this end, mass spectrometric analysis has two main advantages: greatly improved specificity while maintaining sensitivity and ease of multiplexing, measuring multiple analytes at the same time.

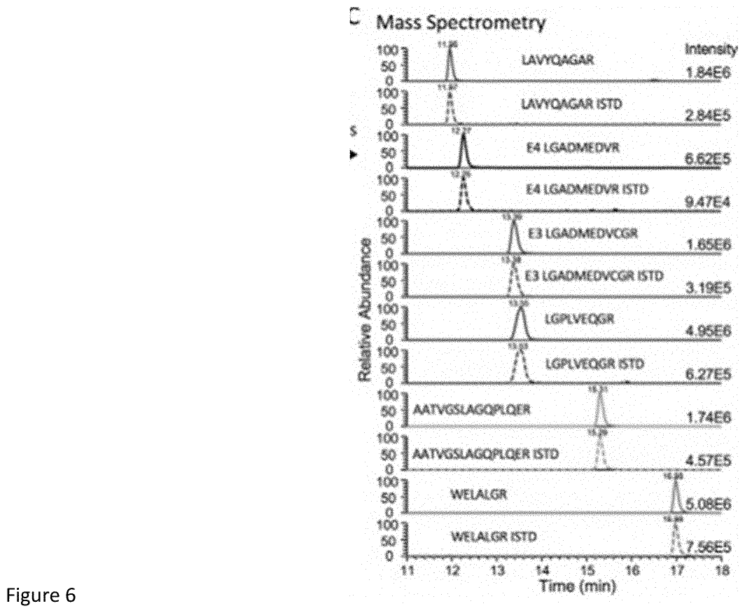

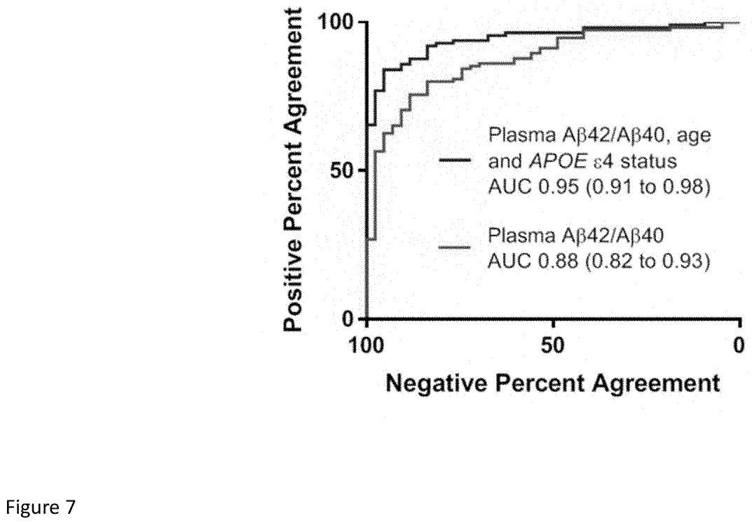

Apolipoprotein E (ApoE) has three major isoforms: ε2, ε3, and ε4, which have single amino acid variations that lead to differences in molecular weight, structure and function. Importantly, a single Apo ε4 allele increases risk of AD 3-4 fold and two Apo ε4 alleles increase AD risk by 10-14 fold (3). Methods have been developed to measure ApoE status by sequencing (genotype measurement) and by liquid chromatography mass spectrometry LC/MS) (phenotype measurement) with 100% concordance (FIG. 6). Sensitivity and specificity of the amyloid-beta blood test substantially improve when ApoE status is combined with age. When we analyzed plasma Aβ42/40 with ApoE status and age, our AUC increased from 88% to 95%, (FIG. 7). Multiplexing Aβ42/40 and ApoE phenotype into a single assay will provide highly sensitive and specific concordance with amyloid PET, while decreasing assay time.

Compare the results of the individual assays and multiplexed assays on 100 blood samples (50 AD and 50 controls); Experimental design: Optimize parameters of multiplexed assay. Pooled CSF and pooled plasma will be used for initial assay development. Existing protocols for Aβ42/40 and ApoE will be multiplexed at various points including immunoprecipitation, protein digestion, solid phase extraction, and liquid chromatography. Results from the multiplexed assays will be compared to the results from the independent assays and the most accurate multiplexed protocol will be chosen. Bioanalytical parameters of the multiplexed assay will be measured and compared to the individual assays. Parameters to be optimized are described above and include analytical sensitivity (LOQ), accuracy, precision, and stability. The multiplexed and individual assays will be run on 100 blood samples (50 amyloid positive by CSF and PET AD vs. 50 cognitive normal, amyloid negative by PET and CSF age matched controls).

### Example 4

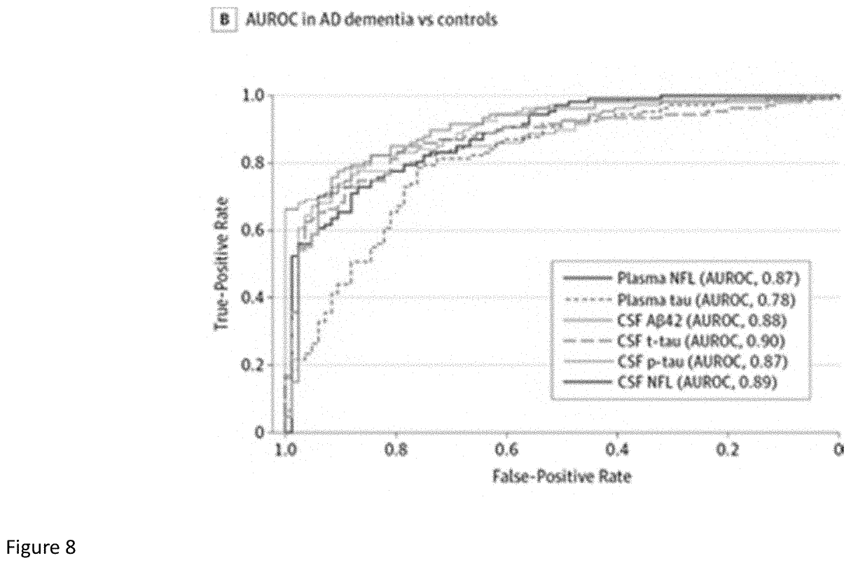

Neurofilament light chain (NfL) is a major component of the cytoskeleton of neurons. Upon neurodegeneration, it is released into the CSF and blood, and can be measured in both as a biomarker of neurodegeneration. Although Nfl is increased in many neurodegenerative diseases, NfL has the potential to aid in staging AD (e.g. asymptomatic years to symptom onset vs. mildly and moderately affected) and in monitoring response to therapeutics during clinical drug trials. A study by Mattsson et. al. (JAMA Neurology, 2017) reports an AUC of 0.87 for plasma NfL differentiating between an AD dementia group and controls (FIG. 8). To date, NfL analysis has predominantly been done by immunoassay. Inclusion of Nfl in our mass spectrometric assay is expected to result in an assay that is more specific for neuronal (and potentially neuron subtypes), sensitive and cost-effective with multiplexing. By building a multiplexed assay with Nfl, additional staging information can be added to the high accuracy blood plasma detection of amyloid plaques.

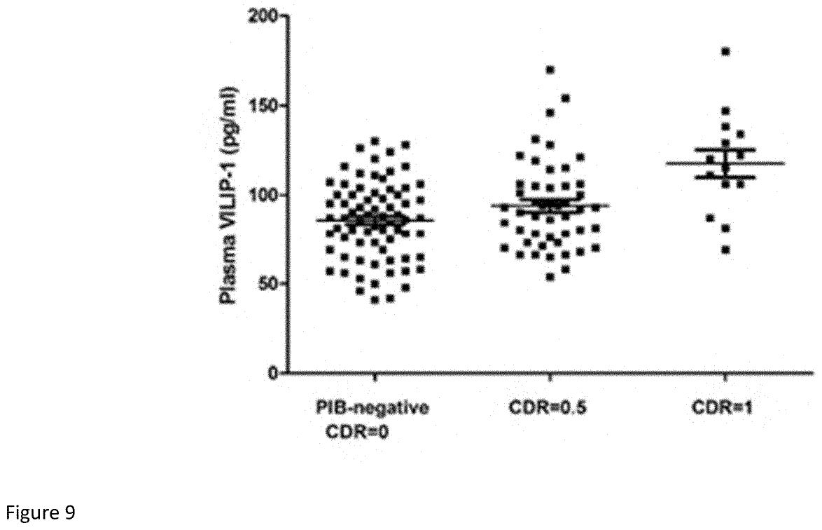

Visinin-like protein one (VILIP-1) is a calcium-sensor neuronal protein that is elevated following neuronal injury. Immunoassay studies by Tarawneh et. al. in both CSF and plasma show separation between the AD group and controls (FIG. 9). Similar to our expectations for NfL, we expect the VILIP-1 mass spectrometry assay to be more specific and precise than the immunoassay and to be amenable to multiplexing.

NfL Assay Development: Antibody development and selection: Recombinant human NfL will be expressed as described by Lewcuk et. al. 2018. Briefly, nucleotides encoding Neurofilament light (NfL) protein amino acids 1-396, (NfL-head+core) will be amplified from a full-length cDNA (RC205920, Origene). The PCR fragments will be purified and cloned into BamHI/EcoRI digested pG (GST) expression plasmid (GE Healthcare). Constructs will be sequenced and transformed into E. coli BL21 (DE3). E. coli BL21(DE3) containing the construct will be cultured in LB media containing ampicillin and protein expression will be induced with isopropyl β-D-1-thiogalactopyranoside (IPTG). Cells will be pelleted via centrifugation and stored at −20° C. pending purification. The pellet will re-suspended in lysis buffer (20 mM Tris, 150 mM NaCl, 1% NP40 pH 7.5) plus complete protease inhibitors (Complete, Roche) and GST-NfL fusion protein will be purified using Glutathione-Sepharose 4B (GE Healthcare). On-bead cleavage of the GST-fusion protein by thrombin will be performed. The cleaved, untagged protein will be eluted with PBS containing protease inhibitor. Monoclonal antibodies against NfL will be generated by immunization of 8-week-old Balb/c mice with the recombinant protein fragments (head+core) using complete Freund's adjuvant (Sigma) as described for generation of tau monoclonal antibodies (Yanamandra et al. 2013). Briefly, after 2-3 dosages with the recombinant protein fragment (approximately 75 μg/mouse), the spleen will be removed and B cells will be fused with the myeloma cell line SP2/0 following standard procedures. Approximately 10 days after fusion, cell media will be screened for NfL antibodies using recombinant protein fragment head+core (amino acids 1-396), and purified bovine NfL protein (Karlsson et al. 1987). Clones that reacted with the recombinant NfL proteins and bovine NfL, but not with a negative control protein will be further grown, subcloned, and subsequently frozen in liquid nitrogen. Reactivity against human NfL will be determined by Western blot from cortex of human brain samples. The isotypes of the antibodies will be determined using a commercially available kit (Pierce Rapid Isotyping Kit-Mouse). Finally, antibodies will be purified using a protein G column (GE Healthcare). Purified NfL antibodies (Table 1) will be compared to commercially available antibodies for their ability to immunoprecipitate NfL from human serum.

TABLE 6
Representative table of antibodies to be
compared for immunoprecipitation of NfL

| | Monoclonal or | | Pruduct |
|---|---|---|---|
| Epitope | Polyclonal | Supplier | number |
| Conserved rod | Monoclonal | UmanDiagnostics | 27017 |
| domain | (UD2) | | |
| | Monoclonal | Abbexa | ABIN6025698 |
| | Monoclonal | Novus Biologicals | ABIN4339158 |
| | (2F11) | | |
| | Monoclonal | Invitrogen | 13-0400 |
| | | Antibodies | |
| Full length | Polyclonal | Abnova | ABIN518281 |
| AA281-396 | Polyclonal | Abbexa | ABIN1176228 |
| AA1-284 | Polyclonal | Synaptic Systems | ABIN1742358 |

Enzyme and peptide selection: Target peptides will be chosen empirically. Recombinant human NfL will be digested with Trypsin or LysN, and analyzed by LC/MS/MS with data dependent acquisition. Candidate peptides will be chosen based on peptide chemistry (lack of oxidation and alkylation sites), retention time, charge states, relative intensity and fragmentation. The top candidates will be analyzed for uniqueness with a BLAST search, then further analyzed in pooled CSF and plasma using a minimum of five technical replicates. Transition ions will be selected from fragmentation of the peptides by collision induced dissociation and the most reproducible fragment ions will be chosen for quantitation. This iterative process will result in selection of several target peptides with two to four transition ions each. Comparison of AD and control CSF and plasma will then be used to determine optimal and specific NFI peptides to use in the final multiplexed assay.

VILIP-1 Assay Development: Antibodies: Immunoassays and monoclonal antibodies against VILIP-1 have been developed. These antibodies, including clone 3A8.1 against epitope S96-Y108, and clone 269.3 against epitope F55-D73, will be compared for their ability to immunoprecipitate VILIP-1 from human plasma.

Enzyme and peptide selection: The same iterative process as described for NfL above will be utilized for VILIP-1 mass spectrometry assay development.

NfL and VILIP-1 are quantified by the mass spectrometry assays and their ability to predict disease state assessed. Prior to analyzing the 50 amyloid positive AD and 50 amyloid negative control samples internal standards, calibration curves, and quality control samples based on our current protocols for plasma Aβ are prepared. Measurement and optimization of analytical sensitivity (LOQ), precision/repeatability, stability, and accuracy of the multiplexed Aβ42/40 & ApoE, NfL, and VILIP-1 assays in blood is completed in accordance with the following.

Internal standard: A stable isotopically labeled “heavy form” of the target protein will be put into each sample at a known concentration. The heavy:light peptide ratios will enable quantitation and a consistent ratio of transition ions and enables highly precise protein quantitation (% CV<2%). Calibration curve: Purified protein will be spiked into synthetic plasma at 6 concentrations ranging from zero to the upper limit of detection and aliquots will be frozen at −80° C. until use. Aliquots will be thawed, the internal standard will be added at a predetermined concentration (same concentration as to be added to QC's and sample), and the sample will be processed as normal. Calibration curves will be run before each sample set and linear fit (y=mx+b). The analytes will be quantitated by comparing the ratio of internal standards to calibration curves. Quality control samples: QC samples are utilized in our lab with amyloid positive AD and amyloid negative cognitively healthy controls. Aliquots from each QC pool will be spiked with internal standard, processed with all test samples and run at the beginning, middle and end of each batch. Acceptability characteristics will be determined for QC material (+/−2SD, % CV<3%) and will be used to detect analytical errors. Any sample with QC outside of two standard deviations will flag an error and will be rerun after the error is corrected. Limit of quantitation (LOQ) and analytical measurement range: We will spike standards into matrix to determine the concentration at which the signal to noise (S/N) is between 10:1 and 20:1 and set that as the lower limit of detection. The linear range will also be determined by this method and will be assessed at +/−20% of the theoretical concentration range. For multiple concentrations over the linear range we will assess peak shape, retention time and ion ratio. Precision/Repeatability and carryover: We will assess 3 levels of QC over 5 days with 5 replicates each day. Carryover will be assessed by including matrix blanks. Acceptable carryover is <20% of lower LOQ. Stability: We will test the stability of samples, internal standards, calibrators, and QCs in solution and in matrix. Short-term stability will be determined at room temperature and long-term stability at −80° C., −20° C., and 4° C. The maximum number of freeze thaw cycles will also be determined. Accuracy: We will compare results from mass spectrometry assays to those from existing immunoassays and assess both the concordance between methods and the individual method's ability to predict disease state.

### Example 5

Experiments were performed to determine if a single immunoprecipitation (IP) could be used to identify both amyloid beta and ApoE without negatively effecting amyloid beta results. One mL of 20 Load 100 samples was used for a normal Abeta IP and for a multiplex IP. Two 0.5 mL IP of these samples, one for normal abeta, one for multiplex, were used. For the multiplex samples, 20% (i.e. 20 uL) of the Formic Acid elution from Abeta IP was removed. The remaining 80% (80 uL) was dried via speed vac, and was processed alongside the aliquot used for normal Abeta analysis. All samples were run in the same Mass Spec queue (on Orbitrap Lumos).

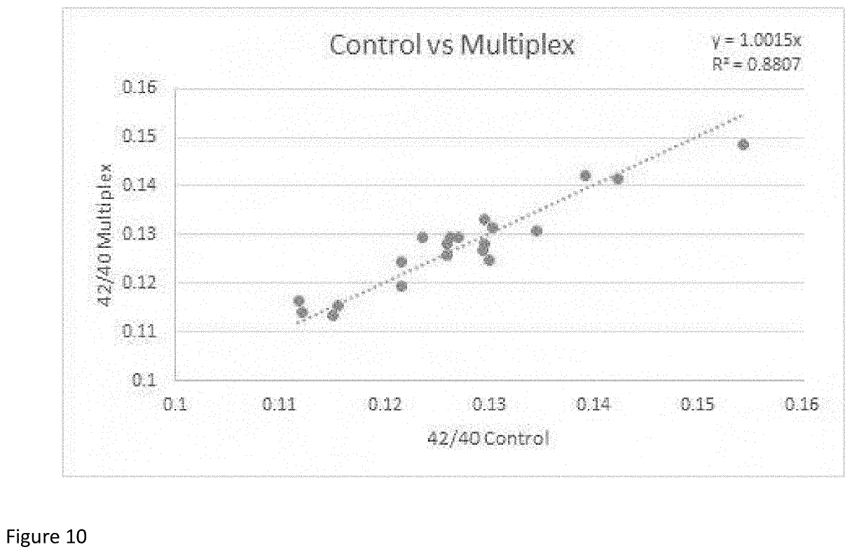

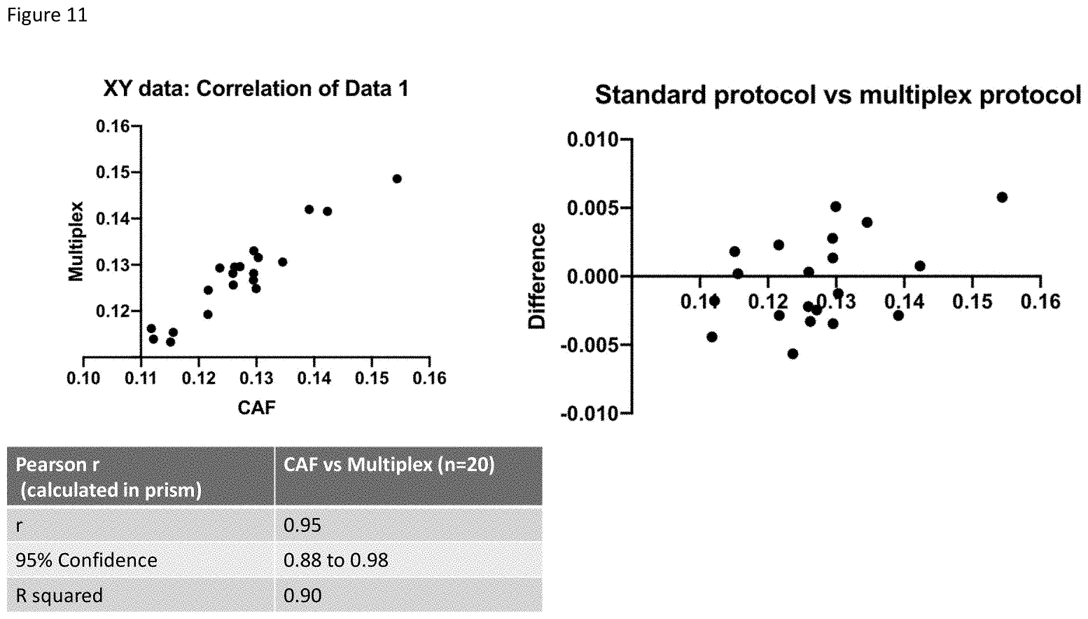

This experiment was used to test the effect of removing 20% of the Abeta IP for ApoE analysis; specifically, to identify whether removing 20% of the sample negatively affected precision, or introduced bias (i.e. positive or negative). The CV of the normal Abeta IP versus the multiplex samples is not more than the CV of duplicate IP's for quality control using the normal methodology. As FIG. 10 and FIG. 11 illustrate, removing 20% of the elution from the standard Abeta IP does not introduce positive or negative bias to the Abeta assay. There is a good correlation between samples processed with normal protocol and samples processed with 20% of the sample removed for ApoE analysis. The precision of the assay was not decreased by removing 20% of the IP for downstream ApoE processing.

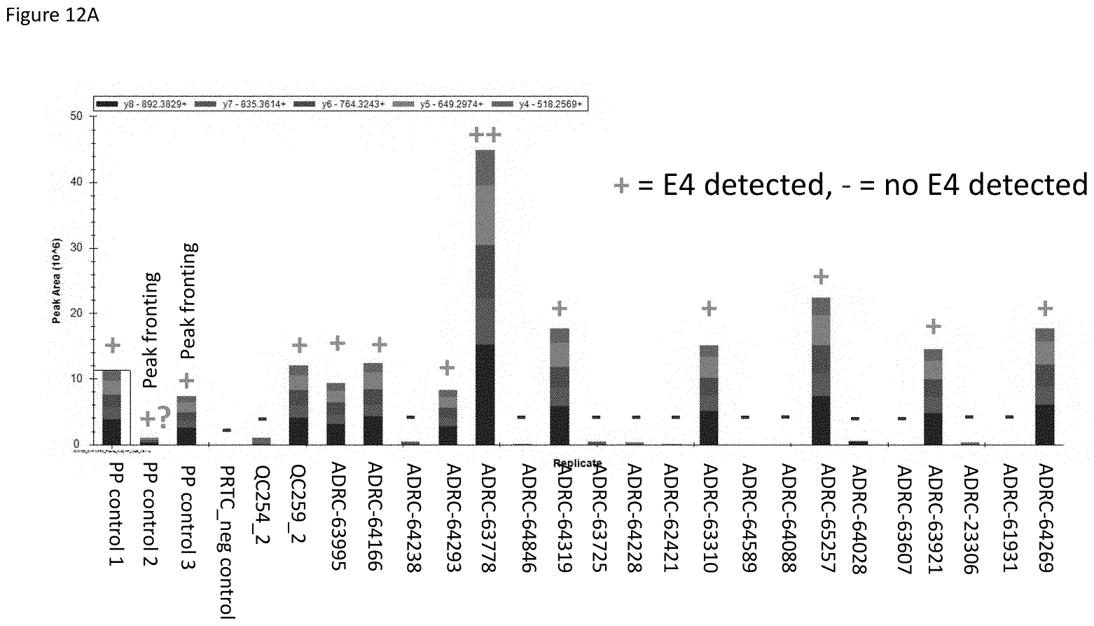

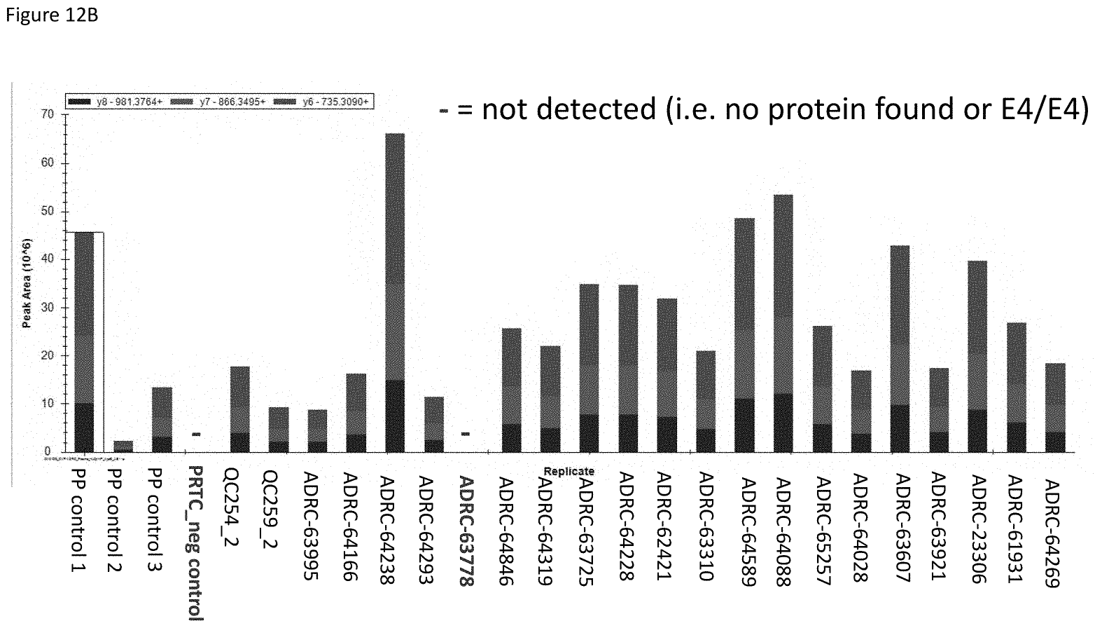

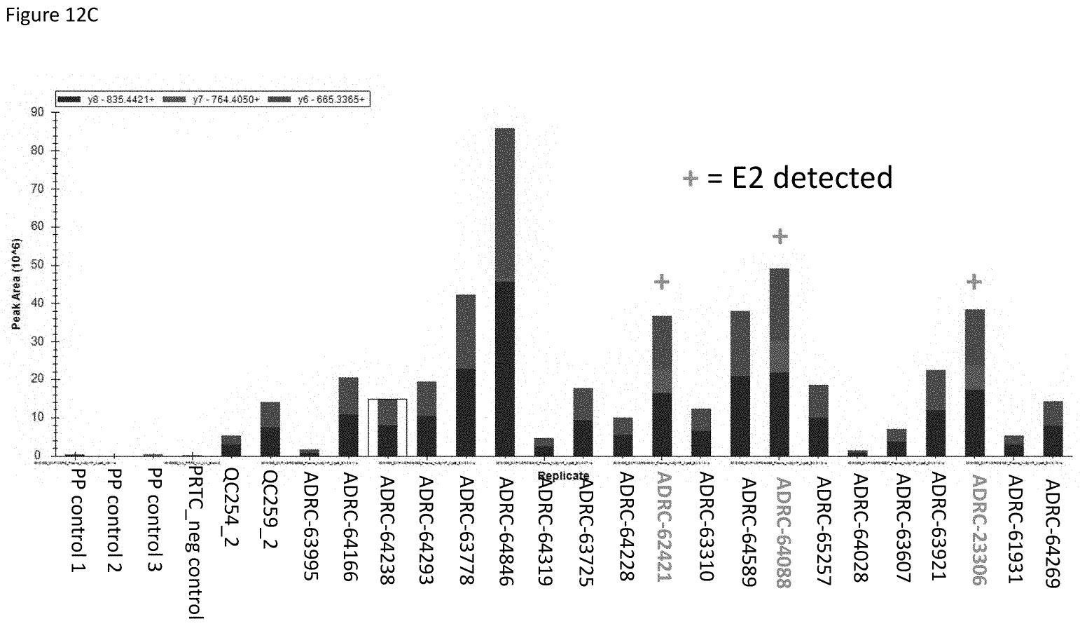

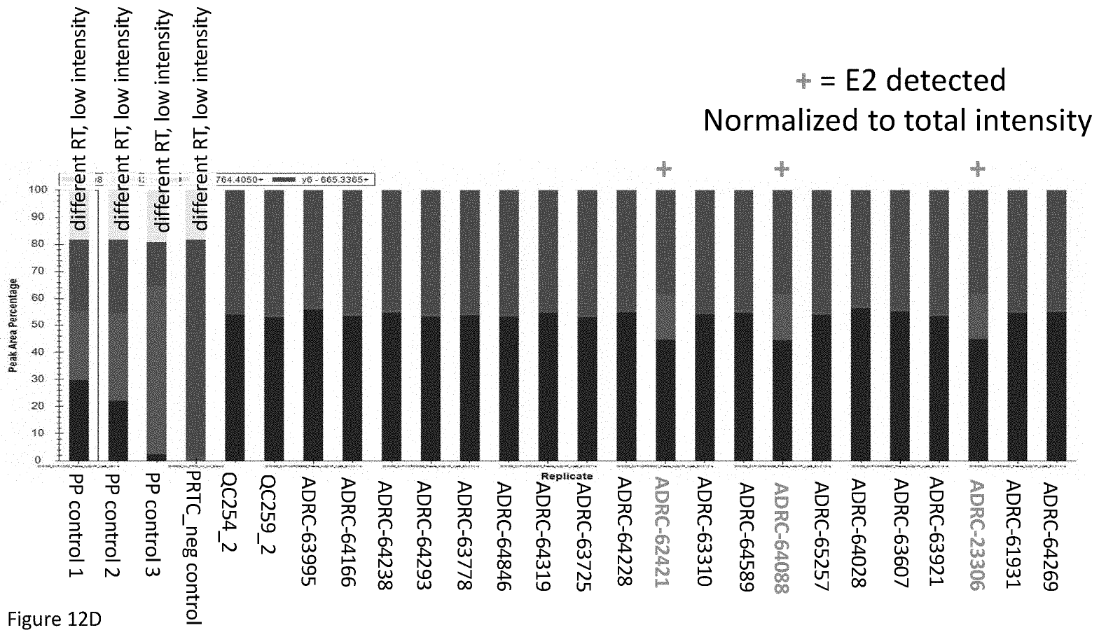

The next question was whether ApoE status could accurately be determined from 20% of the Abeta IP. The 20% that was removed for this experiment was dried, reduced, alkylated, and digested with Trypsin before being analyzed on Xevo. FIG. 12 shows a skyline analysis of each of the 20 samples, low control, high control, and 3 pooled plasma controls. All samples were resuspended in 0.1% FA except PP controls 2 and 3, which were resuspended in 5% CAN. The pooled plasma control 1=ApoE IP (1:500 dilution compared to other samples). Pooled plasma controls 2 and 3 were 10% of Abeta IP, recombined at top tip, and 30% of Abeta IP, recombined at top tip. Samples were resuspended in Abeta resuspension solvent with ACN reduced to 10% (i.e. HSA, 10% FA, 5% ACN).

Results (see FIG. 12) demonstrate that ApoE genotype may be determined from 20% of the Abeta IP with 100% accuracy.

What is claimed is:

1. A method for identifying a subject as a candidate for further diagnostic testing and/or a therapeutic intervention, the method comprising:
(a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and optionally determining the concentration of the marker of neurodegeneration; and;
(b) identifying the subject as a candidate for further diagnostic testing and/or a therapeutic intervention when the Aβ42/Aβ40 value is less than 0.126, and the Aβ42/Aβ40 value is obtained by a system that provides a probability of detecting A13 amyloidosis equal to or greater than about 90%.

2. A method for detecting Aβ amyloidosis, the method comprising:
(a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and optionally determining the concentration of the marker of neurodegeneration; and
(b) identifying the subject as having or at risk of developing A13 amyloidosis when the Aβ42/Aβ40 value is less than 0.126, and the Aβ42/Aβ40 value is obtained by a system that provides a probability of detecting A13 amyloidosis equal to or greater than about 90%.

3. A method for grading a subject for the stage of disease, the method comprising:
(a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and optionally determining the concentration of the marker of neurodegeneration; and
(b) identifying the subject as having or at risk of developing disease when the Aβ42/Aβ40 value is less than 0.126, and the Aβ42/Aβ40 value is obtained by a system that provides a probability of detecting disease equal to or greater than about 90%.

4. A method for treating a subject with Aβ amyloidosis, the method comprising:
(a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and optionally determining the concentration of the marker of neurodegeneration;
(b) identifying the subject as a candidate further diagnostic testing and/or a therapeutic intervention when the Aβ42/Aβ40 value is less than 0.126, and the Aβ42/Aβ40 value is obtained by a system that provides a probability of detecting Aβ amyloidosis equal to or greater than about 90%; and
(c) administering treatment to the diagnosed individual.

5. A method of selecting subjects for a clinical trial for treating Aβ amyloidosis, the method comprising:
(a) detecting an ApoE peptide and measuring the concentration of Aβ42 and Aβ40 and optionally a marker of neurodegeneration in a blood sample obtained from the subject, and then determining ApoE ε4 status, calculating the Aβ42/Aβ40 value and optionally determining the concentration of the marker of neurodegeneration; and
(b) identifying the subject as a candidate for the clinical trial when the Aβ42/Aβ40 value is less than 0.126, and the Aβ42/Aβ40 value is obtained by a system that provides a probability of detecting Aβ amyloidosis equal to or greater than about 90%.

6. The method of any one of the preceding claims, wherein the Aβ42/Aβ40 value is about 0.125 or less.

7. The method of any one of the preceding claims, wherein the Aβ42/Aβ40 value is about 0.124 or less.

8. The method of any one of the preceding claims, wherein the probability of diagnosing the disease is calculated using a receiver operating curve (ROC) area under the curve (AUC).

9. The method of any one of the preceding claims, wherein the predetermined threshold is determined by a data point of the highest specificity at the highest sensitivity on the ROC curve.

10. The method of any one of the preceding claims, wherein the marker of neurodegeneration is selected from one or more of neurofilament light chain, tau isoforms, visinin-like protein 1, and neurogranin isoforms.

11. The method of any one of the preceding claims, wherein the subject (a) was not previously diagnosed with Aβ amyloidosis, (b) is asymptomatic, (c) is a potential participant in a clinical trial for a disease associated with Aβ amyloidosis, (d) is a candidate for amyloid imaging, or (e) any combination of (a) through (d).

12. The method of claim 3 or claim 4, wherein treatment is determined based on the grade of Aβ amyloidosis.

13. The method of claim 4 or claim 12, wherein the treatment is a non-pharmacological treatment, a pharmacological treatment, or treatment with an imaging agent followed by detection of the imaging agent.

14. The method of claim 13, wherein the imaging agent is a functional imaging agent or a molecular imaging agent.

15. The method of claim 12, wherein the treatment is a non-pharmacological treatment.

16. The method of claim 12, wherein the treatment is a pharmacological treatment.

17. The method of claim 12, wherein the treatment is administered through a clinical trial.


SEQUENCE LISTING (7 sequences)

SEQ ID NO: 1 - protein, 9 aa, Homo sapiens
LAVYQAGAR

SEQ ID NO: 2 - protein, 9 aa, Homo sapiens
LGADMEDVR

SEQ ID NO: 3 - protein, 11 aa, Homo sapiens
LGADMEDVCGR

SEQ ID NO: 4 - protein, 9 aa, Homo sapiens
LGPLVEQGR

SEQ ID NO: 5 - protein, 15 aa, Homo sapiens
AATVGSLAGQPLQER

SEQ ID NO: 6 - protein, 7 aa, Homo sapiens
WELALGR

SEQ ID NO: 7 - protein, 9 aa, Homo sapiens
CLAVYQAGR